(19) United States
(12) Patent Application Publication
(10) Pub. No.: US 2008/0227974 A1
(43) Pub. Date: Sep. 18, 2008

(54) Novel Substituted Azetidinones

(75) Inventors: Jeffrey A. Pfefferkorn, Mystic, CT (US); Bharat K. Trivedi, Farmington Hills, MI (US)

Correspondence Address: PFIZER INC., PATENT DEPARTMENT, MS8260-1611, EASTERN POINT ROAD, GROTON, CT, 06340, US

(73) Assignee: Pfizer Inc.

(21) Appl. No.: 11/997,391

(22) PCT Filed: Jul. 20, 2006
(86) PCT No.: PCT/IB06/02130

§ 371 Date: Feb. 20, 2008

Related U.S. Application Data
(60) Provisional application No. 60/704,487, filed on Aug. 1, 2005.

(51) Int. Cl. C07D 205/04

(52) U.S. Cl. 540/200

(57) ABSTRACT

Novel azetidinones and pharmaceutical compositions are described, as are the methods of using such compounds and compositions to treat subjects, including humans, suffering from hyperlipidemia, hypercholesterolemia, hypertriglyceridemia and atherosclerosis.

### FIELD OF THE INVENTION

This invention relates to a group of novel azetidinones. These compounds inhibit cholesterol absorption and are thus useful as hypocholesterolemic agents in the treatment and prevention of atherosclerosis.

### BACKGROUND OF THE INVENTION

Atherosclerotic coronary heart disease represents the major cause of death and cardiovascular morbidity in the western world. Risk factors for atherosclerotic coronary heart disease include hypertension, diabetes mellitus, family history, maleness, smoking and elevated plasma cholesterol. Elevated plasma cholesterol and lipoprotein are significant atherosclerotic risk factors. Thus, a causative link between elevated plasma cholesterol levels, atherosclerosis, and coronary heart disease has been firmly established. Harwood et al., 34 J. Lipid Research 377-378 (1993). More specifically, a total cholesterol level in excess of 225-250 mg/dl is associated with significant elevation of risk.

An increase in low density lipoprotein (LDL) concentration is correlated with increased atherosclerosis. The liver is the major organ responsible for cholesterol biosynthesis and catabolism and the site of synthesis and secretion of very low-density lipoprotein (VLDL) which are subsequently metabolized to LDL. When cholesterol absorption in the intestines is reduced, by whatever means, less cholesterol is delivered to the liver. The consequence of this action is decreased hepatic lipoprotein (VLDL) production and an increase in the hepatic clearance of plasma cholesterol, mostly as LDL. Thus, the net effect of an inhibition of intestinal cholesterol absorption is a decrease in plasma cholesterol levels.

Several 2-azetidinone compounds have been reported as being useful in lowering cholesterol and/or in inhibiting the formation of cholesterol-containing lesions in mammalian arterial walls: U.S. Pat. No. 5,688,785 describes 2-azetidinone compounds wherein the 3-position substituent is arylalkylene or arylalkenylene wherein the alkylene or alkenylene portion is interrupted by a hetero atom, phenylene or cycloalkylene; U.S. Pat. No. 5,698,548 describes 2-azetidinone compounds wherein the 3-position substituent is an arylalkylspirocyclic group; U.S. Pat. Reissue No. RE37721 describes 2-azetidinone compounds wherein the 3-position substituent is an arylalkylene group substituted in the alkylene portion by a hydroxy group; US 2003/0105028 describes glucose-derived conjugates of 2-azetidinone compounds wherein the 1-position substituent is a hydroxyl-substituted phenyl group and the 4-position substituent is a hydroxyphenyl group; and U.S. Pat. No. 5,756,470 discloses 2-azetidinones having an aryl group at the 4-position which is substituted with a hydroxyl and a glucuronide group.

At least one substituted azetidinone, ezetimibe, is currently commercially available for the treatment of hypercholesterolemia. Ezetimibe can be adminstered alone or in combination with other cholesterol reducing modalities. The effectiveness of available antilipidemic therapies is limited, in part because of poor patient compliance due to unacceptable side effects and tolerability as well as minimal efficacy or potency. Furthermore, certain drug products may not be advantageous to all patients because of genetic polymorphisms regarding cholesterol biosynthesis.

For the reasons set forth above, there is a continuing need for novel antilipidemic agents that may be used alone or in combination with other agents that provide increased efficacy and tolerability with decreased toxicity. This invention relates to novel chemical compounds having pharmacological activity, to pharmaceutical compositions which include these compounds, and to pharmaceutical methods of treatment using the compounds.

### SUMMARY OF THE INVENTION

The present invention provides a compound of the formula (1):

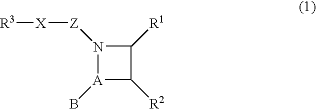

or pharmaceutically acceptable salts, esters, hydrates, amides, or stereoisomers thereof, wherein

### A-B is C═O, C═S, SO, or SO₂;

X is a C₁-C₃ alkylene optionally containing a double or triple bond, or a C₁-C₃ heteroalkylene, wherein the C₁-C₃ alkylene or C₁-C₃ heteroalkylene is unsubstituted or substituted on carbon atoms with 0, 1, or 2 substituents selected from the group consisting of C₁-C₆ alkyl, ═O, —C(O)Rₐ, —OR_{b}, R_{c}, —OC(O)R_{d}, —NR′R″, halo, C₃-C₆ cycloalkyl, C₃-C₆ heterocycloalkyl, aryl, heteroaryl, and cyano; wherein
Rₐ is hydroxy, —OC₁₁C₆ alkyl or C₁-C₆ alkyl;
R_{b} is hydrogen, SO₃H, PO₃H, C₁-C₆ alkyl, or C₁-C₆ aralkyl;
R_{c} is YG; wherein Y is NR′, S, or O;
R_{d} is NR′R″, C₁-C₆ alkyl, C₁-C₆ aralkyl, C₃-C₆ cycloalkyl, C₃-C₆ heterocycloalkyl, aryl, or heteroaryl;
R′ and R″ are each independently selected from the group consisting of hydrogen and C₁-C₆ alkyl;
Z is a C₁-C₂ alkylene optionally substituted with 0, 1, or 2 substituents selected from the group consisting of C₁-C₆ alkyl, ═O, —C(O)Rₐ, —OR_{b}, —OC(O)R_{d}, —NR′R″, halo, C₃-C₆ cycloalkyl, C₃-C₆ heterocycloalkyl, aryl, heteroaryl, and cyano;
R¹ is aryl or heteroaryl optionally substituted with one to three substituents independently selected from the group consisting of halo, —C(O)Rₐ, —OR_{b}, R_{c}, C₁-C₂₀ alkyl, C₁-C₆ aralkyl, and cyano;
R² is C₁-C₆ alkyl, C₃-C₆ cycloalkyl, C₃-C₆ heterocycloalkyl, aryl, heteroaryl, or C₁-C₆ aralkyl, wherein said
C₁-C₆ alkyl, C₃-C₆ cycloalkyl, C₃-C₆ heterocycloalkyl, aryl, heteroaryl, or C₁-C₆ aralkyl groups are optionally substituted with one to three substituents independently selected from the group consisting of halo,
—C(O)Rₐ, —OR_{b}, C₁-C₂₀ alkyl, and cyano;
R³ is C₃-C₆ cycloalkyl, C₃-C₆ heterocycloalkyl, aryl or heteroaryl, wherein the C₃-C₆ cycloalkyl,
C₃-C₆ heterocycloalkyl, aryl or heteroaryl groups are optionally substituted with one to three substituents independently selected from the group consisting of halo, —C(O)Rₐ, —OR_{b}, C₁-C₂₀ alkyl, C₁-C₆ alkyl-NR′R″, and cyano; and
G is selected from the group consisting of hydrogen,

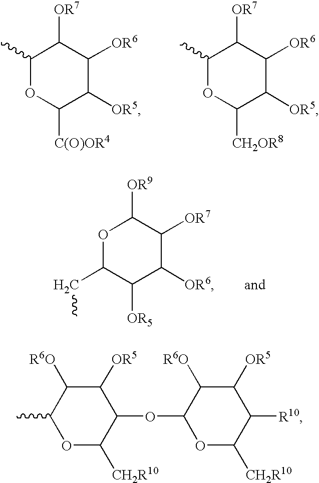

wherein

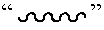

indicates the point of attachment and wherein R⁴, R⁵, R⁶, R⁷, R⁸, and R⁹ are each independently selected from the group consisting of hydrogen, C₁-C₆ alkyl, C₁-C₆ aralkyl, —C(O)C₁₁C₆ alkyl,
—C(O)aryl, and aryl; and R¹⁰ is selected from the group consisting of hydrogen, hydroxy, C₁-C₆ alkyl,
—OC₁₁C₆ alkyl, and NR′R″.

The present invention further provides inter alia the following compounds:
(3R,4R)-bis-(4-methoxy-phenyl)-1-(3-phenyl-propyl)-azetidin-2-one;
(3S,4S)-bis-(4-methoxy-phenyl)-1-(3-phenyl-propyl)-azetidin-2-one;
3R-(4-Fluoro-phenyl)-4R-(4-methoxy-phenyl)-1-(3-phenyl-propyl)-azetidin-2-one;
4-(4-Benzyloxy-phenyl)-3-(4-fluoro-phenyl)-1-phenethyl-azetidin-2-one;
3R-(4-Fluoro-phenyl)-4R-(4-hydroxy-phenyl)-1-phenethyl-azetidin-2-one;
3S-(4-Fluoro-phenyl)-4S-(4-hydroxy-phenyl)-1-phenethyl-azetidin-2-one;
3R-(4-Fluoro-phenyl)-4R-(4-hydroxy-phenyl)-1-(3-phenyl-propyl)-azetidin-2-one;
3S-(4-Fluoro-phenyl)-4S-(4-hydroxy-phenyl)-1-(3-phenyl-propyl)-azetidin-2-one;
3R-(4-Fluoro-phenyl)-4R-(4-hydroxy-phenyl)-1-(4-phenyl-butyl)-azetidin-2-one;
3S-(4-Fluoro-phenyl)-4S-(4-hydroxy-phenyl)-1-(4-phenyl-butyl)-azetidin-2-one;
4-(4-Benzyloxy-phenyl)-3-(4-fluoro-phenyl)-1-(4-phenyl-butyl)-azetidin-2-one;
4R-(4-Benzyloxy-phenyl)-3R-(4-fluoro-phenyl)-1-[3-(4-fluoro-phenyl)-3-O-propyl]-azetidin-2-one;
3-(4-Fluoro-phenyl)-1-[3-(4-fluoro-phenyl)-3-O-propyl]-4-(4-hydroxy-phenyl)-azetidin-2-one;
4R-(4-Benzyloxy-phenyl)-3R-(4-fluoro-phenyl)-1-[3-(4-fluoro-phenyl)-3S-hydroxy-propyl]-azetidin-2-one;
3R-(4-Fluoro-phenyl)-1-[3-(4-fluoro-phenyl)-3S-hydroxy-propyl]-4R-(4-hydroxy-phenyl)-azetidin-2-one;
4S-(4-Benzyloxy-phenyl)-3S-(4-fluoro-phenyl)-1-[3-(4-fluoro-phenyl)-3S-hydroxy-propyl]-azetidin-2-one;
3S-(4-Fluoro-phenyl)-1-[3-(4-fluoro-phenyl)-3S-hydroxy-propyl]-4S-(4-hydroxy-phenyl)-azetidin-2-one;
   or a pharmaceutically acceptable salt, ester, amide, hydrate, or stereoisomer thereof.

The invention still further provides a compound of the formula (2) or (3):

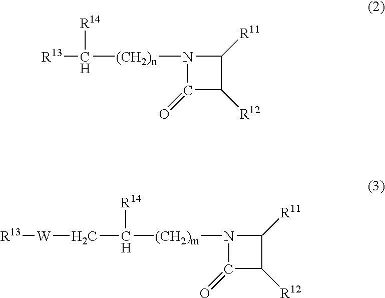

or a pharmaceutically acceptable salt, ester, hydrate, amide, or stereoisomer thereof, wherein
n is 1, 2, 3, or 4;
m is 1 or 2;

### W is O, NR′R″ or S;

R¹¹ is phenyl optionally substituted with one to three substituents independently selected from the group consisting of halo, —C(O)Rₐ, —OR_{b}, R_{c}, C₁-C₂₀ alkyl, C₁-C₆ aralkyl, and cyano;
R¹² is C₁-C₆ alkyl, C₃-C₆ cycloalkyl, C₃-C₆ heterocycloalkyl, aryl, heteroaryl, or C₁-C₆ aralkyl, wherein said C₁-C₆ alkyl, C₃-C₆ cycloalkyl, C₃-C₆ heterocycloalkyl, aryl, heteroaryl, or C₁-C₆ aralkyl groups are optionally substituted with one to three substituents independently selected from the group consisting of halo,
—C(O)Rₐ, —OR_{b}, C₁-C₂₀ alkyl, and cyano;
R¹³ is aryl or heteroaryl, wherein the aryl or heteroaryl groups are optionally substituted with one to three substituents independently selected from the group consisting of halo, —C(O)Rₐ, —OR_{b}, C₁-C₂₀ alkyl, and C₁-C₆ alkyl-NR′R″; and
R¹⁴ is selected from the group consisting of C₁-C₆ alkyl, ═O, C(O)Rₐ, OR_{b}, R_{c}, OC(O)R_{d}, —NR′R″, halo,
C₃-C₆ cycloalkyl, C₃-C₆ heterocycloalkyl, aryl, heteroaryl, and cyano; wherein
Rₐ is hydroxy, —OC₁₁C₆ alkyl or C₁-C₆ alkyl;
R_{b} is hydrogen, SO₃H, PO₃H, C₁-C₆ alkyl, or C₁-C₆ aralkyl;
R_{c} is YG; wherein Y is NR′, S, or O;
R_{d} is NR′R″, C₁-C₆ alkyl, C₁-C₆ aralkyl, C₃-C₆ cycloalkyl, C₃-C₆ heterocycloalkyl, aryl, or heteroaryl;
R′ and R″ are each independently selected from the group consisting of hydrogen and C₁-C₆ alkyl;
G is selected from the group consisting of hydrogen,

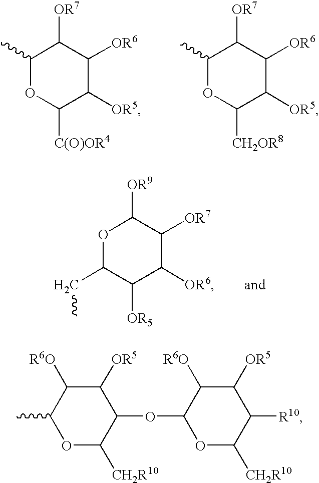

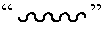

indicates the point of attachment, R⁴, R⁵, R⁶, R⁷, R⁸, and R⁹ are each independently selected from the group consisting of hydrogen, C₁-C₆ alkyl, C₁-C₆ aralkyl, —C(O)C₁-C₆ alkyl, —C(O)aryl, and aryl; and R¹⁰ is selected from the group consisting of hydrogen, hydroxy, C₁-C₆ alkyl, —OC₁-C₆ alkyl, and NR′R″.

The invention still further provides a pharmaceutical composition comprising a compound of formula (1), (2), or (3) and a pharmaceutically acceptable carrier, diluent, solvent or vehicle.

The invention still further provides a method of treating a subject suffering from hyperlipidemia, hypercholesterolemia, hypertriglyceridemia or atherosclerosis, comprising administering a therapeutically effective amount of a compound of formula (1) to the subject in need thereof.

The invention still further provides a combination comprising a compound of formula (1), (2), or (3) and a pharmaceutically active agent.

### DETAILED DESCRIPTION OF THE INVENTION

The invention provides a novel azetidinone of formula (1), as defined above, or a pharmaceutically acceptable salt, ester, hydrate, amide, or stereoisomer thereof, wherein A-B, X, Rₐ, R_{b}, R_{c}, R_{d}, R′, R″, Z, R¹, R², R³, G, R⁴, R⁵, R⁶, R⁷, R⁸, R⁹ and R¹⁰ are as defined above.

The invention further provides a compound of formula (1), as defined above, wherein R¹ is aryl optionally substituted with one to three substituents independently selected from the group consisting of halo, —C(O)Rₐ, —OR_{b}, R_{c}, C₁-C₆ alkyl, and C₁-C₆ aralkyl, wherein Rₐ, R_{b}, and R_{c} are as defined above.

The invention further provides a compound of formula (1), as defined above, wherein R¹ is aryl optionally substituted with one to three substituents independently selected from the group consisting of halo, —C(O)Rₐ, —OR_{b}, R_{c}, C₁-C₆ alkyl, and C₁-C₆ aralkyl, and R² is aryl optionally substituted with one to three substituents independently selected from the group consisting of halo, —C(O)Rₐ, —OR_{b}, and C₁-C₆ alkyl, wherein Rₐ, R_{b}, and R_{c} are as defined above.

The invention further provides a compound of formula (1), as defined above, wherein R¹ is aryl optionally substituted with one to three substituents independently selected from the group consisting of halo, —C(O)Rₐ, —OR_{b}, R_{c}, C₁-C₆ alkyl, and C₁-C₆ aralkyl, and R² is aryl optionally substituted with one to three substituents independently selected from the group consisting of halo, —C(O)Rₐ, —OR_{b}, and C₁-C₆ alkyl, and R³ is aryl optionally substituted with one to three substituents independently selected from the group consisting of halo, —C(O)Rₐ, —OR_{b}, C₁-C₆ alkyl, and C₁-C₆ alkyl-NR′R″, wherein Rₐ, R_{b}, R_{c}, R′, and R″ are as defined above.

The invention further provides a compound of formula (1), as defined above, wherein A-B is C═O;

X is a C₁-C₃ alkylene or C₁-C₃ heteroalkylene, wherein the C₁-C₃ alkylene or C₁-C₃ heteroalkylene is optionally substituted on carbon atoms with 0, 1, or 2 substituents selected from the group consisting of C₁-C₆ alkyl, ═O, —C(O)Rₐ, —OR_{b}, —OC(O)R_{d}, and halo;
Z is a C₁-C₂ alkylene optionally substituted with 0, 1, or 2 substituents selected from the group consisting of C₁-C₆ alkyl, ═O, C(O)Rₐ, OR_{b}, OC(O)R_{d}, halo, aryl, heteroaryl, and NR′R″;
R¹ is aryl optionally substituted with one to three substituents independently selected from the group consisting of halo, —C(O)Rₐ, —OR_{b}, R_{c}, C₁-C₆ alkyl, and C₁-C₆ aralkyl;
R² and R³ are each independently aryl optionally substituted with one to three substituents independently selected from the group consisting of halo, —C(O)Rₐ, —OR_{b}, and C₁-C₆ alkyl; wherein Rₐ, R_{b}, R_{c}, R_{d}, R′ and R″ are as defined above.

The invention further provides a compound of formula (1), as defined above, wherein

### A-B is C═O;

X is a C₁-C₃ alkylene or a C₁-C₃ heteroalkylene, wherein the C₁-C₃ alkylene or C₁-C₃ heteroalkylene is unsubstituted or substituted on carbon atoms with 0, 1, or 2 substituents selected from the group consisting of C₁-C₆ alkyl, ═O, —C(O)Rₐ, —OR_{b}, —OC(O)R_{d}, and halo;
Z is a C₁-C₂ alkylene optionally substituted with 0, 1, or 2 substituents selected from the group consisting of C₁-C₆ alkyl, ═O, halo, C(O)Rₐ, OR_{b}, OC(O)R_{d}, and aryl;
R¹ is aryl optionally substituted with one to three substituents independently selected from the group consisting of halo, —C(O)Rₐ, —OR_{b}, R_{c}, C₁-C₆ alkyl, and C₁-C₆ aralkyl;
R² and R³ are each independently aryl optionally substituted with one to three substituents independently selected from the group consisting of halo, —C(O)Rₐ, —OR_{b}, and C₁-C₆ alkyl; wherein Rₐ, R_{b}, R_{c}, R_{d}, R′, and R″ are as defined above.

The invention further provides a compound of formula (1), as defined above, wherein

### A-B is C═O;

X is a C₁-C₃ alkylene that is optionally substituted with 0, 1, or 2 substituents selected from the group consisting of C₁-C₆ alkyl, ═O, —C(O)Rₐ, —OR_{b}, —OC(O)R_{d}, and halo;
Z is a C₁-C₂ alkylene optionally substituted with 0, 1, or 2 substituents selected from the group consisting of C₁-C₆ alkyl, ═O, and OR_{b};
R¹ is aryl optionally substituted with one to three substituents independently selected from the group consisting of halo, —C(O)Rₐ, —OR_{b}, R_{c}, C₁-C₆ alkyl, and C₁-C₆ aralkyl;
R² and R³ are each independently aryl optionally substituted with one to three substituents independently selected from the group consisting of halo, —C(O)Rₐ, —OR_{b}, and C₁-C₆ alkyl, wherein Rₐ, R_{b}, R_{c}, R_{d}, R′ and R″ are as defined above.

The invention further provides a compound of formula (1), as defined above, wherein

### A-B is C═O;

X is a C₁-C₃ alkylene that is optionally substituted with 0, 1, or 2 substituents selected from the group consisting of C₁-C₆ alkyl, ═O, and —OR_{b};
Z is a C₁-C₂ alkylene optionally substituted with 0, 1, or 2 substituents selected from the group consisting of ═O and OR_{b};
R¹ is phenyl optionally substituted with —OR_{b}; R² is phenyl optionally substituted with a substituent independently selected from the group consisting of halo and —OR_{b}; and R³ is phenyl optionally substituted with halo and —OR_{b}, wherein R_{b} is as defined above.

The present invention further provides a compound of the formula (2) or (3), as defined above, or a pharmaceutically acceptable salt, ester, hydrate, amide, or stereoisomer thereof, wherein

n, m, W, R¹¹, R¹², R¹³, R¹⁴, Rₐ, R_{b}, R_{c}, R_{d}, R′, R″, G, R⁴, R⁵, R⁶, R⁷, R⁸, R⁹, and R¹⁰ are as defined above.

The invention further provides a compound of formula (2) or (3), as defined above, wherein R¹² is phenyl optionally substituted with one to three substituents independently selected from the group consisting of halo, —C(O)Rₐ, —OR_{b}, and C₁-C₆ alkyl, wherein Rₐ and R_{b} are as defined above.

The invention further provides a compound of formula (2) or (3), as defined above, wherein R¹³ is phenyl optionally substituted with one to three substituents independently selected from the group consisting of halo, —C(O)Rₐ, —OR_{b}, C₁-C₆ alkyl, and C₁-C₆ alkyl-NR′R, wherein R′, R″, Rₐ, and R_{b} are as defined above.

The invention further provides a compound of formula (2) or (3), as defined above, wherein R¹² is phenyl optionally substituted with one to three substituents independently selected from the group consisting of halo, —C(O)Rₐ, —OR_{b}, and C₁-C₆ alkyl, and R¹³ is phenyl optionally substituted with one to three substituents independently selected from the group consisting of halo, —C(O)Rₐ, —OR_{b}, C₁-C₆ alkyl, and C₁-C₆ alkyl-NR′R, wherein R′, R″, Rₐ, and R_{b} are as defined above.

The invention further provides a pharmaceutical composition comprising a compound of formula (1), (2) or (3) and a pharmaceutically acceptable carrier, diluent, solvent or vehicle.

The invention further provides a method of treating a subject suffering from hyperlipidemia, hypercholesterolemia, hypertriglyceridemia or atherosclerosis, comprising administering a therapeutically effective amount of a compound of formula (1), (2), or (3) to the subject in need thereof.

The invention still further provides a combination comprising a compound of formula (1), (2), or (3) and a pharmaceutically active agent.

The invention further provides a combination comprising a compound a formula (1), (2), or (3) and a pharmaceutically active agent, wherein the pharmaceutically active agent is a CETP inhibitor, a PPAR-activator, an MTP/Apo B secretion inhibitor, HDL-cholesterol raising agent, HMG-CoA reductase inhibitor, triglyceride lowering agent, a cholesterol synthesis inhibitor, a cholesterol modulating agent, a fibrate, niacin, an ion-exchange resin, an antioxidant, an ACAT inhibitor, bile acid sequestrant, an anti-hypertensive agent, or an acetylcholine esterase inhibitor.

The invention further provides a combination comprising a compound of formula (1), (2), or (3) and an HMG-CoA reductase inhibitor wherein the HMG-CoA reductase inhibitor is a statin.

The invention further provides a combination comprising a compound a formula (1), (2), or (3) and a pharmaceutically active agent, wherein the pharmaceutically active agent is a CETP inhibitor, a PPAR-activator, an MTP/Apo B secretion inhibitor, HDL-cholesterol raising agent, HMG-CoA reductase inhibitor, triglyceride lowering agent, a cholesterol synthesis inhibitor, a cholesterol modulating agent, a fibrate, niacin, an ion-exchange resin, an antioxidant, an ACAT inhibitor, bile acid sequestrant, an anti-hypertensive agent, or an acetylcholine esterase inhibitor and a pharmaceutically acceptable carrier, diluent, solvent, or vehicle.

The present invention further includes each of the title compounds set forth in the Examples herein.

As used above, and throughout the specification, the following terms, unless otherwise indicated, shall be understood to have the following meanings.

The article “a” or “an” as used herein refers to both the singular and plural form of the object to which it refers.

The following definitions apply regardless of whether a term is used by itself or in combination with other terms, unless otherwise indicated. Therefore, the definition of “alkyl” applies to “alkyl” as well as the “alkyl” portions of “hydroxyalkyl”, “haloalkyl”, “alkoxy”, “aralkyl”, etc. The definition of “aryl” applies to “aryl” as well as the “aryl” portions of “heteroaryl”, “aralkyl”, “arylthio”, etc.

The term “alkyl” as used herein refers to a linear or branched hydrocarbon of from 1 to 20 carbon atoms. Non-limiting examples include methyl, ethyl, n-propyl, isopropyl, n-butyl, sec-butyl, isobutyl, tert-butyl, n-pentyl, n-hexyl, n-decyl, tetradecyl, and the like.

The alkyl can be optionally substituted and the term “substituted alkyl” means that the alkyl group is substituted by one or more substituents independently selected from the group consisting of halo, aryl, cycloalkyl, nitro, cyano, hydroxy, lower alkoxy, lower thioalkoxy, amino, —C(O)C₁-C₆ alkyl, —C—OH, C₁-C₆ alkyl, —OSO₃H, —OPO₃H, —OC₁-C₆ alkyl,

—O-aryl, ═O, ═S, —SH, —CO₂H, —CO₂C₁-C₆ alkyl, —NR′R″, —N⁺R′R″R′″T⁻, —NR′SO₂R″, —R′C(O)NR′R″, or
—C(O)NR′R″, where R′, R″, and R′″ are each independently hydrogen, C₁-C₆ alkyl, cycloalkyl, alkenyl, alkynyl, aryl, aralkyl, heteroaryl, heteroaralkyl, where N, R′ and R″, or N, R′, and R′″, or N, R″ and R′″, or N, R′, R″, and R′″ may be joined together to form a 4-7 member monocyclic or bicyclic ring optionally containing at least one additional heteroatom selected from N, O and S that can also be optionally substituted with at least one to three of the substituents recited for the term alkyl; where T is a representative counter anion forming a pharmaceutically acceptable salt, such as for example, bromide, chloride, sulfate, nitrate, bisulfate, acetate, oxalate, benzoate, tartrate, fumarate, and the like.

The term “lower alkyl” as used herein refers to a subset of alkyl which means a linear or branched hydrocarbon radical having from 1 to 6 carbon atoms. Non-limiting examples include methyl, ethyl, n-propyl, isopropyl, n-butyl, sec-butyl, isobutyl, tert-butyl, n-pentyl, n-hexyl, and the like. Alternatively, lower alkyl is referred to as “C₁-C₆ alkyl.” The lower alkyl group can also be substituted with at least one to three of the substituents as previously recited for the term alkyl.

The term “alkoxy” as used herein refers to an alkyl-O— group in which the alkyl group is as previously defined. Useful alkoxy groups can comprise 1 to 12 carbon atoms. The term “lower alkoxy” means an alkyl-O— group in which the alkyl group comprises 1 to 6 carbon atoms. Non-limiting examples of a lower alkoxy include methoxy, ethoxy, isopropoxy, and the like. The alkyl group of the alkoxy is linked to an adjacent moiety through the ether oxygen.

The term “alkenyl” as used herein means a linear or branched hydrocarbon radical from 2 to 12 carbon atoms having at least one carbon-carbon double bond. Non-limiting examples of an alkenyl include ethenyl, 1-propenyl, 1-butenyl, 2-butenyl, 2-pentenyl, 3-methyl-3-butenyl, 1-hexenyl, 3-heptenyl, 1-octenyl, 1-nonenyl, 1-decenyl, 1-undecenyl, 1-dodecenyl, and the like. The alkenyl group may be optionally substituted with at least one to three of the substituents as previously recited for the term alkyl.

The term “alkynyl” as used herein means a linear or branched hydrocarbon radical from 2 to 12 carbon atoms having at least one carbon-carbon triple bond. Non-limiting examples include 3-propynyl,

1-butynyl, 3-pentynyl, 3-methyl-3-butynyl, 1-hexynyl, 3-heptynyl, 1-octynyl, 1-nonynyl, 1-decynyl, and the like. The alkynyl group may be optionally substituted with at least one to three of the substituents as previously recited for the term alkyl.

The term “aryl” as used herein refers to a C₅-C₁₄ mono-, bi- or polycarbocyclic aromatic ring system which is optionally substituted by at least one substituent selected from alkyl, lower alkoxy, lower thioalkoxy, halogen, —CO₂H, —CO₂(C₁-C₆)alkyl, —C(O)C₁-C₆ alkyl, —OSO₃H, —OPO₃H, or —OC₁-C₆ alkyl,

—O(CH₂)₀₋₂CF₃, —O-aryl, —OSO₂R′, nitro, cyano —OH, —SH, —CF₃, —NR′R″, —NR′SO₂R″, —NR′C(O)NR′R″,
—S(O)₁₋₂ alkyl, S(O)₁₋₂aryl, —SO₂NR′R″, or —C(O)NR′R″, where R′, and R″ are independently hydrogen, C₁-C₆ alkyl, cycloalkyl, alkenyl, alkynyl, aryl, aralkyl, heteroaryl, or heteroaralkyl, or N, R′ and R″ may be joined together to form a 4-7 member monocyclic or bicyclic ring optionally containing at least one additional heteroatom selected from N, O and S, Non-limiting examples of aryl include phenyl, naphthyl, indenyl, 2-chlorophenyl, 3-chlorophenyl, 4-chlorophenyl, 2-methylphenyl, 3-methylphenyl, 4-methylphenyl, 2-methoxyphenyl, 3-methoxyphenyl, 4-methoxyphenyl, 2-chloro-3-methylphenyl, 2-chloro-4-methylphenyl, 2-chloro-5-methylphenyl, 3-chloro-2-methylphenyl, 3-chloro-4-methylphenyl, 4-chloro-2-methylphenyl, 4-chloro-3-methylphenyl, 5-chloro-2-methylphenyl, 2,3-dichlorophenyl, 2,5-dichlorophenyl, 3,4-dichlorophenyl, 2,3-dimethylphenyl, 3,4-dimethylphenyl, and the like. The aryl group may be optionally substituted with at least one to three “ring system substituents” which may be the same or different, and are as defined below.

The term “aralkyl” as used herein means an aryl-alkyl group, in which the aryl and alkyl groups are as previously defined. Linkage to the rest of the molecule may be through either the aryl or alkyl portion of the aralkyl moiety. The aralkyl group may be optionally substituted by at least one to three substituents as recited above for alkyl and aryl. Non-limiting examples of aralkyl include benzyl, phenethyl, naphthlenylmethyl, tolyl, and the like.

The term “aralkenyl” as used herein means an aryl-alkenyl group in which the aryl and alkenyl groups are as previously defined. The aralkenyl group may be optionally substituted with one to three substituents as recited above for aryl and alkenyl. Non-limiting examples of aralkenyl include 2-phenethenyl, 2-naphthylethenyl, and the like.

The term “alkylene” as used herein refers to a divalent group derived from a linear or branched chain saturated hydrocarbon having from 1 to 10 carbon atoms by the removal of two hydrogen atoms. The preferred alkylene refers to a linear or branched hydrocarbon chain diradical having from 1 to 3 carbon atoms. The alkylene group may be optionally substituted with one or more of the substituents recited for the term alkyl, and selected from lower alkoxy, lower thioalkoxy, —O(CH₂)₀₋₂CF₃, halo, nitro, cyano, ═O, ═S, —OH, —SH, —CF₃, —CO₂H, —CO₂C₁-C₆ alkyl, —NR′R″, or —C(O)NR′R″, where R′ and R″ are independently hydrogen, C₁-C₆ alkyl, cycloalkyl, alkenyl, alkynyl, aryl, aralkyl, heteroaryl, heteroaralkyl, or N, R′ and R″ may be joined together to form a 4-7 member monocyclic or bicyclic ring optionally containing at least one additional heteroatom selected from N, O and S. Useful alkylene groups have from 1 to 6 carbon atoms (C₁-C₆ alkylene). Non-limiting examples of alkylene include methylene (—CH₂—), ethylene (—CH₂CH₂—), propylene (—(CH₂)₃—), and the like.

The term “aroyl” means an aryl-C(O)— group in which the aryl group is as previously defined. Non-limiting examples of aroyl include benzoyl, 1-naphthoyl, 2-naphthoyl, and the like.

The term “acyl”, as used herein means an HC(O)— or alkyl-C(O)— in which the alkyl group is as previously defined. Preferred acyls contain a lower alkyl. Non-limiting examples of acyl include formyl, acetyl, propanoyl, 2-methylpropanoyl, butanoyl, and the like.

The term “aryloxy”, as used herein means an aryl-O— in which the aryl group is as previously defined. Non-limiting examples of aryloxy include phenoxy, naphthoxy, and the like.

The term “arylthio”, as used herein means an aryl-S— in which the aryl group is as previously described. Non-limiting examples of arylthio include phenylthio, heptylthio, and the like.

The term “aralkylthio” as used herein means an aralkyl-S— group in which the aralkyl is as previously defined. Non-limiting examples of aralkylthio include benzylthio, 2-phenyl-ethanethiol, and the like.

The term “alkoxycarbonyl”, as used herein means an alkoxy-C(O)— in which the alkoxy is as previously defined. Non-limiting examples of alkoxycarbonyl include methoxycarbonyl, ethoxycarbonyl, and the like.

The term “aryloxycarbonyl”, as used herein means an aryl-O—C(O)— group in which the aryl group is as previously described. Non-limiting examples of aryloxycarbonyl include phenoxycarbonyl, naphthoxycarbonyl, and the like.

The term “aralkoxycarbonyl”, as used herein means an aralkyl-O—C(O)— group in which the aralkyl group is as previously defined. Non-limiting examples of aralkoxycarbonyl include benzyloxycarbonyl, and the like

The term “alkylsulfonyl”, as used herein means an alkyl-S(O)₂— in which the alkyl group is as previously defined. Preferred groups are those in which the alkyl group is lower alkyl.

The term “alkylsulfinyl”, as used herein means an alkyl-S(O)— group. Preferred groups are those in which the alkyl group is lower alkyl.

The term “arylsulfonyl”, as used herein means an aryl-S(O)₂— group.

The term “arylsulfinyl”, as used herein means an aryl-S(O)— group.

The term “cycloalkyl”, as used herein refers to a saturated cyclic C₃-C₁₂ alkyl group, where alkyl is as previously defined. Non-limiting examples of cycloalkyl include cyclopropyl, cyclobutyl, cyclopentyl, cyclohexyl, cycloheptyl, cycloctyl, decalinyl, norpinanyl, or adamantyl. The cycloalkyl group may be optionally substituted with at least one of those substituents recited above for alkyl or alkylene. Non-limiting examples of substituted cycloalkyl groups include fluorocyclopropyl, 2-iodocyclobutyl, 2,3-dimethylcyclopentyl, 2,2-dimethoxycyclohexyl, 3-phenylcyclopentyl, and the like.

The term “cycloalkenyl”, as used herein refers to a saturated cyclic C₃-C₁₂ alkenyl group having at least one carbon-carbon double bond, where alkenyl is as previously defined. Nonlimiting examples of cycloalkenyl include cyclopropene, cyclopentene, cyclopenta-1-3-diene, cyclohexene, cycloheptene, cyclohepta-1-4-diene, and the like.

The term “hydrocarbon chain”, as used herein refers to a linear hydrocarbon of from 1 to 12 carbon atoms. The hydrocarbon chain is optionally substituted with one or more substituents selected from alkyl, alkoxy, thioalkoxy, —O(CH₂)₀₋₂CF₃, halogen, nitro, cyano, ═O, ═S, —OH, —SH, —CF₃, —CO₂H, —CO₂C₁-C₆ alkyl, —NR′R″, —C(O)NR′R″, —N⁺R′R″R′″T⁻, —NR′S(O)₂R″, or —R′C(O)NR′R″, where R′, R″, and R′″ are independently hydrogen, C₁-C₆ alkyl, cycloalkyl, alkenyl, alkynyl, aryl, aralkyl, heteroaryl, heteroaralkyl, or N, R′ and R″, or N, R′, and R′″, or N, R″ and R′″ or N, R′, R″, and R′″ may be joined together to form a 4-7 member monocyclic or bicyclic ring optionally containing at least one additional heteroatom selected from N, O and S.

The term “halogen” or “halo”, as used herein means fluorine or fluoro, chlorine or chloro, bromine or bromo or iodine or iodo.

The term “heteroatom”, as used herein means oxygen (O), nitrogen (N), or sulfur (S) as well as sulfoxyl or sulfonyl (S(O) or SO₂) unless otherwise indicated.

The term “heteroaryl”, as used herein means an aryl group, as previously defined, containing one or more heteroatoms, as previously defined. The heteroaryl may be optionally substituted with at least one of the substituents previously recited for “aryl”. Non-limiting examples of heteroaryl include thienyl, benzothienyl (2-benzothienyl, 3-benzothienyl, and the like), indolizinyl, pyrazinyl, furanyl, benzofuranyl, pyrrolyl, pyridyl, pyrimidinyl (2-pyrimidinyl, 4-pyrimidinyl, and the like), imidazolyl (1-imidazolyl, 2-imidazolyl, and the like), benzimidazolyl (1-benzimidazolyl, 2-benzimidazolyl, and the like), triazolyl (1-triazolyl, 3-triazolyl, and the like), isothiazolyl, pyrazolyl (1-pyrazolyl, 3-pyrazolyl, 4-pyrazolyl, and the like), oxazolyl (2-oxazolyl, 4-oxazolyl, and the like), benzoxazolyl (2-benzoxazolyl, 4-benzoxazolyl and the like), tetrazolyl (1-tetrazolyl, 3-tetrazolyl, and the like), thiazolyl (2-thiazolyl, 4-thiazolyl, and the like), indolyl (1-indolyl, 2-indolyl, and the like), isoindolyl (1-isoindolyl, 2-isoindolyl, and the like), quinazolinyl, quinolinyl (2-quinolinyl, 3-quinolinyl, and the like), isoquinolinyl (3-isoquinolinyl, 5-isoquinolinyl, and the like).

The term “heterocycle”, as used herein means a saturated mono-, bi- or polycyclic ring containing one or more heteroatoms selected from N, O, and S. The heterocycle may be optionally substituted with at least one of those substituents recited above for alkyl. Non-limiting examples of heterocycle include piperidinyl, pyrrolidinyl, I-piperazinyl, 2-piperazinyl, 2-morpholinyl, 4-morpholinyl, piperazinyl, azetidinyl, aziridinyl, thietanyl, and the like.

The term “heterocyclenyl”, as used herein means a non-aromatic monocyclic or multicyclic ring system of about 3 to about 12 ring atoms, preferably about 5 to about 10 ring atoms, in which one or more of the atoms in the ring system is/are element(s) other than carbon, for example nitrogen, oxygen, or sulfur atoms, and which contains at least one carbon-carbon double bond or carbon-nitrogen double bond. The prefix aza, oxa, or thia before heterocyclenyl means that at least a nitrogen, oxygen, or sulfur atom, respectively, is present as a ring atom. Non-limiting examples of heterocyclenyl include 1,2,3,4-tetrahydropyridine, 2-pyrrolinyl, 2-imidazolinyl, 1,2-dihydropyridyl, and the like.

The term “heteroaralkyl”, as used herein means heteroaryl-alkyl, in which heteroaryl and alkyl are both as previously defined. Linkage to the rest of the molecule can be either through the heteroaryl or the alkyl portion of the heteroaralkyl moiety. The heteroaralkyl may be optionally substituted with at least one of those substituents previously recited for alkyl and heteroaryl. Nonlimiting examples of heteroarylalkyl include 2-propyl-pyridine, 3,4-methyl-1H-pyrrole, and the like.

The term “heteroaralkenyl”, as used herein means heteroaryl-alkenyl, in which heteroaryl and alkenyl are both as previously defined. Linkage to the rest of the molecule can be either through the heteroaryl or the alkenyl portion of the heteroaralkenyl moiety. The heteroaralkenyl may be optionally substituted with at least one of those substituents previously recited for alkenyl and heteroaryl. Non-limiting examples of heteroaralkenyl include 2-(pyrid-3-yl)ethenyl, 2-(quinolin-3-yl)ethenyl, and the like.

The term “heterocycloalkyl”, as used herein means heterocycle-alkyl, in which the heterocycle and the alkyl are both as previously defined. Linkage to the rest of the molecule can be either through the heterocycle or the alkyl portion of the heterocycloalkyl moiety. The heterocycloalkyl may be optionally substituted with at least one of those substituents recited above for alkyl and heterocycle. Non-limiting examples of heterocycloalkyl include 2-methyl piperidine, 2-ethyl-5-methyl-pyrrolidine, and the like.

The term “thioalkyl” or “alkylthio” means an alkyl-S— in which the alkyl group is a previously defined. The alkyl is linked to an adjacent moiety through the sulfinyl moiety. Non-limiting examples of thioalkyl include methylthio, ethylthio, isopropylthio, and the like.

The term “thioalkoxy” means an alkoxy-S— in which the alkoxy group is a s previously defined. The alkoxy is linked to an adjacent moiety throught the sulfinyl moiety. The term “lower thioalkoxy” means an alkyl-O—S— group in which the alkyl group comprises 1 to 6 carbon atoms. Non-limiting examples of thioalkoxy include methoxysulfanyl, ethoxysulfanyl, and the like.

The term “ring” as used herein includes heteroaryl, heterocycle, cycloalkyl and aryl, each as previously defined, and further includes fused, monocyclic, bicyclic, and polycyclic permutations thereof.

“Ring system substituent” means a substituent attached to an aromatic or non-aromatic ring system which, for example, replaces hydrogen on the ring system. Ring system substituents may be the same or different, each being independently selected from the group consisting of alkyl, aryl, heteroaryl, aralkyl, aralkenyl, heteroaralkyl, heteroaralkenyl, hydroxy, alkoxy, aryloxy, aralkoxy, acyl, halo, nitro, cyano, carboxy, alkoxycarbonyl, aryloxycarbonyl, aralkoxycarbonyl, aroyl, alkylsulfonyl, arylsulfonyl, heteroarylsulfonyl, alkylsulfinyl, arylsulfinyl, heteroarylsulfinyl, alkylthio, arylthio, heteroarylthio, aralkylthio, heteroaralkylthio, cycloalkyl, cycloalkenyl, heterocyclo, heterocyclenyl, heterocycloalkyl, and NR′R″, wherein R′ and R″ are each independently H, C₁-C₆ alkyl, cycloalkyl, alkenyl, alkynyl, aryl, aralkyl, heteroaryl, or heteroaralkyl, or N, R′, and R″ may be joined together to form a 4-7 member monocyclic or bicyclic ring optionally containing at least one additional heteroatom selected from N, O and S.

The term “stereoisomer” as used herein refers to both geometric (e.g., cis and trans isomers) and/or optical isomers (e.g., R and S enantiomers) of a compound of the invention. Racemic, enantiomeric, diastereomeric, and epimeric mixtures of isomers are contemplated by the present invention. Compounds of formula 1, 2, or 3 containing one or more asymmetric carbon atom can exist as two or more stereoisomers. Where a compound of formula 1, 2, or 3 contains an alkenyl or alkenylene group, geometric cis/trans isomers are possible. Where structural isomers are interconvertible via a low energy barrier, tautomeric isomerism (‘tautomerism’) can occur. This can take the form of proton tautomerism in compounds of formula 1, 2, or 3 containing, for example, an imino, keto, or oxime group, or so-called valence tautomerism in compounds which contain an aromatic moiety. It follows that a single compound may exhibit more than one type of isomerism. Accordingly, included within the scope of the present invention are all stereoisomers and tautomeric forms of the compounds of formula 1, 2, or 3, including compounds exhibiting more than one type of isomerism, and mixtures of one or more thereof. Also included are acid addition or base salts wherein the counterion is optically active, for example, d-lactate or 1-lysine, or racemic, for example, dl-tartrate or dl-arginine.

Cis/trans isomers may be separated by conventional techniques well known to those skilled in the art, for example, chromatography and fractional crystallization.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC).

Alternatively, the racemate (or a racemic precursor) may be reacted with a suitable optically active compound, for example, an alcohol, or, in the case where the compound of formula 1, 2, or 3 contains an acidic or basic moiety, a base or acid such as 1-phenylethylamine or tartaric acid. The resulting diastereomeric mixture may be separated by chromatography and/or fractional crystallization and one or both of the diastereoisomers converted to the corresponding pure enantiomer(s) by means well known to a skilled person.

Chiral compounds of the invention (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% by volume of isopropanol, typically from 2% to 20%, and from 0 to 5% by volume of an alkylamine, typically 0.1% diethylamine. Concentration of the eluate affords the enriched mixture.

The term “racemate” as used herein, is meant to include both the racemic compound wherein one homogeneous form of crystal is produced containing both enantiomers in equimolar amounts and the racemic mixture or conglomerate wherein two forms of crystal are produced in equimolar amounts each containing the single enantiomer. Such mixtures may be separated by conventional techniques known to those skilled in the art—see, for example, Stereochemistry of Organic Compounds by E. L. Eliel and S. H. Wilen (Wiley, New York, 1994).

When a bond to a substituent is shown to cross the bond(s) connecting 2 atoms in a ring, then such substituent may be bonded to any atom in the ring, provided the atom will accept the substituent without violating its valency. When there appears to be several atoms of the substituent that may bond to the ring atom, then it is the first atom of the listed substituent that is attached to the ring, unless indicated otherwise.

Unless indicated otherwise, “compound of the invention” or “compounds of the invention” includes the compound itself as well as pharmaceutically acceptable salts, esters, amides, hydrates, or stereoisomers thereof.

The term “patient” or “subject” means all animals and mammals, including humans. Examples of patients or subjects include humans, cows, dogs, cats, goats, sheep, pigs, and rabbits.

The phrases “effective amount” and “therapeutically effective amount” mean that amount of a compound of Formula 1, 2, or 3, and other pharmacological or therapeutic agents described below, that will elicit a biological or medical response in a tissue, system, animal, or mammal that is being sought by the administrator (such as a researcher, doctor, or veterinarian) which includes alleviation of the symptoms of the condition or disease being treated and the prevention, slowing or halting of progression of one or more conditions, for example vascular conditions such as hyperlipidemia, atherosclerosis, hypercholesterolemia, hypertriglyceridemia, sitosterolemia, vascular inflammation, and the like. As would be understood by a skilled artisan, a “therapeutically effective amount” will vary from subject to subject and will be determined on a case by case basis. Factors to consider include, but are not limited to, the subject being treated, weight, health, and compound administered.

The term “a pharmaceutically acceptable salt, ester, amide, hydrate, or stereoisomer” as used herein refers to those acid addition salts, base addition salts, esters, amides, hydrates, and stereoisomers (optical, geometric, and tautomeric) of the compounds of the present invention which are, within the scope of sound medical judgment, suitable for use in contact with the tissues of patients without undue toxicity, irritation, allergic response, and the like, commensurate with a reasonable benefit/risk ratio, and effective for their intended use, as well as the zwifterionic forms, where possible, of the compounds of the invention.

Further, the term “a pharmaceutically acceptable salt” refers to the relatively non-toxic, inorganic and organic acid addition or base salts of compounds of the invention. These salts can be prepared in situ during the final isolation and purification of the compounds or by separately reacting the purified compound in its free form with a suitable organic or inorganic acid or base and isolating the salt thus formed. Representative anionic or acid addition salts include acetate, aspartate, besylate, bicarbonate, carbonate, camysylate, citrate, edisylate, fumarate, gluconate, hydrobromide, bromide, hydrochloride, chloride, D-lactate, L-lactate, malate, mesylate, pamoate, phosphate, succinate, sulphate, D-tartrate, L-tartrate, benzoate, gluceptate, glucuronate, hibenzate, isethionate, malonate, methylsulphate, 2-napsylate, nicotinate, nitrate, orotate, stearate, tosylate, adipate, arabogalactanesulphate, ascorbate, estolate, galacturonate, glutamate, hippurate, 3-hydroxy-2-naphthoate, 1-hydroxy-2-naphthoate, iodide, lactobionate, maleate, mandelate, mucate, napadisylate, oleate, oxalate, saccharate, salicylate, sulphosalicylate, cholate, and tryptophanate. (See, for example, Berge S. M., et al., “Pharmaceutical Salts,” J. Pharm. Sci., 1977; 66:1-19, which is incorporated herein by reference.) The free base form may be regenerated by contacting the salt form with a base. While the free base may differ from the salt form in terms of physical properties, such as solubility, the salts are equivalent to their respective free bases for the purposes of the present invention.

Representative cationic or base salts include calcium, choline, magnesium, potassium, sodium, aluminum, ammonium, quaternary ammonium, and amine cations including tetramethylammonium, tetraethylammonium, methylamine, dimethylamine, trimethylamine, triethylamine, ethylamine, and the like, arginine, benzathine, diethylamine, diolamine, glycine, lysine, meglumine, olamine, tromethamine (Tris), 2-amino-2-methylpropan-1-ol, benethamine, erbumine (tert-butylamine), epolamine (hydroxyethylpyrrolidine), ethylenediamine, hydrabamine, morpholine, piperazine, procaine, silver, trolamine, zinc, adenine, arginine, cytosine, glucosamine, guanidine, guanine, nicotinamide, ornithine, praline, pyridoxine, serine, tyrosine, and valine. Hemisalts, for example, hemicalcium may also be formed.

Examples of pharmaceutically acceptable, non-toxic esters of the compounds of the invention include C₁-C₆ alkyl esters wherein the alkyl group is a linear or branched chain. Acceptable esters also include C₅-C₇ cycloalkyl esters as well as aralkyl esters such as, but not limited to, benzyl. C₁-C₄ alkyl esters are preferred. Esters of the compounds of the present invention may be prepared according to conventional methods.

Examples of pharmaceutically acceptable, non-toxic amides of the compounds of the invention include amides derived from ammonia, primary (C₁-C₆)alkyl amines and secondary di-(C₁-C₆)alkyl amines wherein the alkyl groups are linear or branched chain. In the case of secondary amines, the amine may also be in the form of a 5- or 6-membered heterocycle containing one nitrogen atom. Amides derived from ammonia, C₁-C₃ alkyl primary amines and C₁-C₂ dialkyl secondary amines are preferred. Amides of the compounds of the invention may be prepared according to conventional methods.

Certain compounds of the present invention can exist in unsolvated form as well as solvated form including hydrated form. In general, the solvated form including hydrated form is equivalent to the unsolvated form and is intended to be encompassed within the scope of the present invention.

The use of prodrugs is contemplated by the present invention. “Prodrugs” are intended to include any covalently bonded carrier which releases the active parent drug according to Formula 1, 2, or 3, in vivo. Further, the term “prodrug” refers to compounds that are transformed in vivo to yield the parent compound of the above formulae, for example, by hydrolysis in blood. A thorough discussion is provided in T. Higuchi and V. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, both of which are hereby incorporated by reference. Examples of prodrugs include acetates, formates, benzoate derivatives of alcohols, and amines present in compounds of Formula 1, 2, or 3.

The compounds of the present invention are suitable to be administered to a patient or subject for the treatment of hyperlipidemia, hypercholesterolemia, hypertriglyceridemia, and atherosclerosis. The compounds of the present invention can be administered to a patient/subject alone, or with another compound of the invention, or as part of a pharmaceutical composition.

A pharmaceutical composition of the invention contains at least one compound of the invention and at least one pharmaceutically acceptable carrier, diluent, solvent or vehicle. The pharmaceutically acceptable carrier, diluent, solvent or vehicle may be any such carrier known in the art including those described in, for example, Remington's Pharmaceutical Sciences, Mack Publishing Co., (A. R. Gennaro edit. 1985). A pharmaceutical composition of the invention may be prepared by conventional means known in the art including, for example, mixing at least one compound of the invention with a pharmaceutically acceptable carrier.

The compounds, compositions, and treatments of the present invention can be administered by any suitable means which produce contact of these compounds with the site of action in the body, for example, in the plasma, liver, rectum, or small intestine of an animal or mammal. Compositions of compounds of the invention are contemplated herein. A composition of the invention can be administered to a patient/subject either orally, rectally, parenterally (intravenously, intramuscularly, or subcutaneously), intracisternally, intravaginally, intraperitoneally, intravesically, locally (powders, ointments, or drops), or as a buccal or nasal spray.

Compositions suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil), and injectable organic esters such as ethyl oleate. Proper fluidity can be maintained, for example, by the use of a coating such as lecithin, by the maintenance of the required particle size in the case of dispersions and by the use of surfactants.

These compositions may also contain additives such as preserving, wetting, emulsifying, and dispensing agents. Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride, and the like. Prolonged absorption of the injectable pharmaceutical form can be brought about by the use of agents delaying absorption, for example, aluminum monostearate and gelatin.

Solid dosage forms for oral administration include capsules, tablets, pills, powders, and granules. In such solid dosage forms, the active compound is admixed with at least one (a) inert customary excipient (or carrier) such as sodium citrate or dicalcium phosphate; (b) fillers or extenders, as for example, starches, lactose, sucrose, glucose, mannitol, and silicic acid; (c) binders, as for example, carboxymethylcellulose, alignates, gelatin, polyvinylpyrrolidone, sucrose, and acacia; (d) humectants, as for example, glycerol; (e) disintegrating agents, as for example, agar-agar, calcium carbonate, potato or tapioca starch, alginic acid, certain complex silicates, and sodium carbonate; (f) solution retarders, as for example paraffin; (g) absorption accelerators, as for example, quaternary ammonium compounds; (h) wetting agents, as for example, cetyl alcohol and glycerol monostearate; (i) adsorbents, as for example, kaolin and bentonite; and (j) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid compositions of a similar type may also be employed as fillers in soft and hard-filled gelatin capsules using such excipients as lactose or milk sugar as well as high molecular weight polyethyleneglycols, and the like.

Solid dosage forms such as tablets, dragees, capsules, pills, and granules can be prepared with coatings and shells, such as enteric coatings and others well-known in the art. They may contain opacifying agents, and can also be of such composition that they release the active compound or compounds in a certain part of the intestinal tract in a delayed manner. Examples of embedding compositions which can be used are polymeric substances and waxes. The active compounds can also be in micro-encapsulated form, if appropriate, with one or more of the above-mentioned excipients.

Liquid dosage forms for oral administration include pharmaceutically acceptable emulsions, solutions, suspensions, syrups, and elixirs. In addition to the active compounds, the liquid dosage forms may contain inert diluents commonly used in the art, such as water or other solvents, solubilizing agents and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular, cottonseed oil, groundnut oil, corn germ oil, olive oil, castor oil and sesame oil, glycerol, tetrahydrofurfuryl alcohol, polyethyleneglycols and fatty acid esters of sorbitan or mixtures of these substances, and the like. Besides inert diluents, compositions include additives, such as, for example, wetting agents, emulsifying and the pending agents, sweetening, flavoring, and perfuming agents, or mixtures thereof. Suspensions, in addition to the active compounds, may contain suspending agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances.

Compositions for rectal administrations are preferably suppositories which can be prepared by mixing the compounds of the present invention with suitable non-irritating excipients or carriers such as cocoa butter, polyethyleneglycol, or a suppository wax, which are solid at ordinary temperatures but liquid at body temperature and therefore, melt in the rectum or vaginal cavity and release the active component.

Dosage forms for topical administration of a compound of this invention include ointments, powders, sprays, and inhalants. The active component is admixed under sterile conditions with a physiologically acceptable carrier and any preservatives, buffers, or propellants as may be required. Ophthalmic formulations, eye ointments, powders, and solutions are also contemplated as being within the scope of this invention.

The compounds of the present invention can be administered to a patient at dosage levels in the range of about 0.1 to about 2,000 mg per day. For a normal human adult having a body weight of about 70 kilograms, a dosage in the range of about 0.01 to about 100 mg per kilogram of body weight per day is preferable. The specific dosage used, however, can vary from patient to patient. For example, the dosage can depend on a numbers of factors including the requirements of the patient, the severity of the condition being treated, and the pharmacological activity of the compound being used. The determination of optimum dosages for a particular patient is well known to those skilled in the art.

The term “treating” or “treatment” refers to curative, palliative and prophylactic treatment, including reversing, ameliorating, alleviating, inhibiting the progress of, or preventing the disorder or condition to which such term applies, or one or more symptoms of such disorder or condition.

The compounds of the invention, as described herein, may be used either alone or in combination with another pharmaceutically active agent described herein, in the treatment of the following diseases/conditions: dyslipidemia, hypercholesterolemia, hypertriglyceridemia, atherosclerosis, peripheral vascular disease, cardiovascular disorders, angina, ischemia, cardiac ischemia, stroke, myocardial infarction, reperfusion injury, angioplastic restenosis, hypertension, diabetes and vascular complications of diabetes, obesity, unstable angina pectoris, Alzheimer's Disease, BPH, osteoporosis, cerebrovascular disease, coronary artery disease, ventricular dysfunction, cardiac arrhythmia, pulmonary vascular disease, renal-vascular disease, renal disease, vascular hemostatic disease, autoimmune disorders, pulmonary disease, sexual dysfunction, cognitive dysfunction, cancer, organ transplant rejection, psoriasis, endometriosis, and macular degeneration. A combination of the invention may be part of a pharmaceutical composition further containing a pharmaceutically active carrier, diluent, solvent or vehicle, each as described herein.

Examples of a suitable pharmaceutically active agent include a CETP inhibitor, a PPAR— activator, an MTP/Apo B secretion inhibitor, HDL-cholesterol raising agent, triglyceride lowering agent, a cholesterol synthesis inhibitor, a cholesterol modulating agent, a fibrate, niacin, an ion-exchange resin, an antioxidant, an ACAT inhibitor, or bile acid sequestrant; an anti-hypertensive agent; an acetylcholine esterase inhibitor, an anti-diabetic compound, an anti-obesity compound, a thyromimetic agent, an anti-resorptive agent, an anti-osteoporosis agent, an antihypertensive agent, or a drug for the treatment of Alzheimer's disease. Specific examples of each of these agents include those known in the art as well as those specified below.

In combination therapy treatment, both the compounds of the invention and the other drug therapies are administered to mammals by conventional methods. The following discussion more specifically describes the various combination aspects of this invention.

Any cholesterol ester transfer protein (“CETP”) inhibitor known in the art that inhibits the transfer of cholesteryl ester and triglyceride between lipoprotein particles, including high density lipoprotein (HDL), low density lipoprotein (LDL), very low density lipoprotein (VLDL), and chylomicrons may be used. The effect of a CETP inhibitor on lipoprotein profile is believed to be anti-atherogenic. Such inhibition may be determined by means known in the art (e.g., Crook et al. Arteriosclerosis 10, 625, 1990; U.S. Pat. No. 6,140,343). Examples of suitable CETP inhibitors include, but are not limited to, those described in U.S. Pat. Nos. 6,197,786, 6,723,752 and 6,723,753. Additional examples of useful CETP inhibitors include the following compounds: [2R, 4S]-4-[(3,5-bis-trifluoromethyl-benzyl)-methoxycarbonyl-amino]-2-ethyl-6-trifluoromethyl-3,4-dihydroxycarbonyl-amino]-2-ethyl-6-trifluoromethyl-3,4-dihydro-2H-quinoline-1-carboxylic acid ethyl ester (Torcetrapib™), and 3-{[3-(4-Chloro-3-ethyl-phenoxy)-phenyl]-[3-(1,1,2,2-tetrafluoro-ethoxy)-benzyl]-amino}-1,1,1-trifluoro-propan-2-ol. To address the poor solubility of many of the CETP inhibitors, an appropriate dosage form such as one comprising (1) a solid amorphous dispersion comprising a cholesteryl ester transfer protein (CETP) inhibitor and an acidic concentration-enhancing polymer; and (2) an acid-sensitive HMG-CoA reductase inhibitor, may be necessary. This dosage form is more fully described in U.S. Ser. No. 10/739,567.

Any peroxisome proliferator activated receptor (“PPAR”) activator known in the art that activates or otherwise interacts with a human PPAR may be used. Three mammalian PPARs have been isolated and termed PPAR-alpha, PPAR-gamma, and PPAR-beta (also known as NUC1 or PPAR-delta). These PPARs regulate expression of target genes by binding to DNA sequence elements, termed PPAR response elements. These elements have been identified in the enhancers of a number of genes encoding proteins that regulate lipid metabolism suggesting that PPARs play a pivotal role in the adipogenic signaling cascade and lipid homeostasis. PPAR-gamma receptors are associated with regulation of insulin sensitivity and blood glucose levels. PPAR-α activators are associated with lowering plasma triglycerides and LDL cholesterol. PPAR-β activators have been reported to both increase HDL-C levels and to decrease LDL-C levels. Thus, activation of PPAR-β alone, or in combination with the simultaneous activation of PPAR-α and/or PPAR-gamma may be desirable in formulating a treatment for dyslipidemia in which HDL is increased and LDL lowered. PPAR-activation is readily determined by those skilled in the art by the standard assays (e.g. US 2003/0225158 and US 2004/0157885). Examples of suitable PPAR-activator compounds include, but are not limited to, those described in US 2003/0171377, US 2003/0225158, US 2004/0157885, and U.S. Pat. No. 6,710,063. Additional examples of useful PPAR-activator compounds include the following compounds: [5-Methoxy-2-methly-4-(4′-trifluoromethly-biphenyl-4-ylmethylsulfanyl)-phenoxy]-acetic acid; [5-Methoxy-2-methyl-4-(3′-trifloromethly-biphenyl-4-ylmethylsulfanyl)-phenoxy]-acetic acid; [4-(4′Fluoro-biphenyl-4-ylmethylsulfanyl)-5-methoxy-2-methyl-phenoxy]-acetic acid; {5-Methoxy-2-methyl-4-[4-(4-trifluoromethyl-benzyloxy)-benzylsulfanyl]-phenoxy}-acetic acid; {{5-Methoxy-2-methyl-4-[4-(5-trifluoromethyl-pryidin-2-yl)-benzylsulfanyl]-phenoxy}-acetic acid; (4-(4-[2-(3-Fluoro-phenyl)-vinyl]-benzylsulfanyl}-5-methoxy-2-methyl-phenoxy)-acetic acid; [5-Methoxy-2-methyl-4-(3-methyl-4′-trifluoromethyl-biphenyl-4-ylmethylsulfanyl)-phenoxy]-acetic acid; [5-Methoxy-2-methyl-4-(4′-trifluoromethyl-biphenyl-3-ylmethylsulfanyl)-phenoxy]-acetic acid; {5-Methoxy-2-methyl-4-[2-(4-trifluoromethyl-benzyloxy)-benzylsulfanyl]-phenoxy}acetic acid; 3-{5-[2-(−5-Methyl-2 phenyl-oxazol-4-yl-ethoxy]-indol-1-yl}-propionic acid; 3-{4[2-(5-methyl-2-phenyl-1,3-oxazol-4-yl)ethoxy-1H-indazol-1-yl}propanoic acid; 2-Methyl-2-{3-[({2-(5-methyl-2-phenyl-1,3-oxazol-4-yl)ethoxy]carbonyl}amino)methyl]phenoxy}propionic acid; 1-{3′-[2-5-Methyl-2-phenyl-1,3-oxazol-4-y]-1,1′-biphenyl-3-yl}oxy)cyclobutanecarboxylic acid; 3-[3-(1-Carboxy-1-methyl-ethoxy)-phenyl]-piperidine-1-carboxylic acid 3-trifluoromethyl-benzyl ester; 2-{2-methyl-4-[({4-methyl-2-[4-(trifluoromethyl)phenyl]-1,3-thiazol-5-yl}methyl)sulfanyl]phenoxy}acetic acid; 2-{2-methyl-4-[({4-methyl-2-[4-(trifluoromethyl)phenyl]-1,3-oxazol-5-yl}methyl)sulfanyl]phenoxy}acetic acid; methyl 2-{4-[({4-methyl-2-[4-(trifluoromethyl)phenyl]-1,3-thiazol-5-yl}methyl)sulfanyl]phenoxy}acetate; 2-{4-[({4-methyl-2-[4-(trifluoromethyl)phenyl]-1,3-thiazol-5-yl}methyl)sulfanyl]phenoxy}acetic acid; (E)-3-[2-methyl-4-({4-methyl-2-[4-(trifluoromethyl)phenyl]-1,3-thiazol-5-yl}methoxy)phenyl]-2-propenoic acid; 2-{3-chloro-4-[({4-methyl-2-[4-(trifluoromethyl)phenyl]-1,3-thiazol-5-yl}methyl)sulfanyl]phenyl}acetic acid; 2-{2-methyl-4-[({4-methyl-2-[3-fluoro-4-(trifluoromethyl)phenyl]-1,3-thiazo 1-5-yl}methyl)sulfanyl]phenoxy}acetic acid; and pharmaceutically acceptable salts thereof.

Any MTP/Apo B secretion (microsomal triglyceride transfer protein and/or apolipoprotein B secretion) inhibitor known in the art which inhibits the secretion of triglycerides, cholesteryl ester and phospholipids may be used. Such inhibition may be readily determined according to standard assays (e.g., Wetterau, J. R. 1992; Science 258:999). Examples of suitable a MTP/Apo B secretion inhibitor include, but are not limited to, imputapride (Bayer) as well as those described in WO 96/40640 and WO 98/23593.

Any ACAT inhibitor known in the art that inhibits the intracellular esterification of dietary cholesterol by the enzyme acyl CoA: cholesterol acyltransferase may be used. Such inhibition may be determined readily according to standard assays, such as the method of Heider et al. described in Journal of Lipid Research. 24:1127 (1983). Examples of suitable ACAT inhibitors include, but are not limited to, those described in U.S. Pat. No. 5,510,379 (carboxysulfonates), WO 96/26948 and WO 96/10559 (urea derivatives). Additional examples include Avasimibe (Pfizer), CS-505 (Sankyo) and Eflucimibe (Eli Lilly and Pierre Fabre).

Any lipase inhibitor (e.g., pancreatic lipase inhibitor, a gastric lipase inhibitor) known in the art that inhibits the metabolic cleavage of dietary triglycerides into free fatty acids and monoglycerides may be used. Such lipase inhibition activity may be readily determined according to standard assays (e.g., Methods Enzymol. 286: 190-231). Examples of a suitable lipase inhibitor include, but are not limited to, lipstatin, (2S,3S,5S,7Z,10Z)-5-[(S)-2-formamido-4-methyl-valeryloxy]-2-hexyl-3-hydro-xy-7,10-hexadecanoic acid lactone, and tetrahydrolipstatin (orlistat), (2S,3S,5S)-5-[(S)-2-formamido-4-methyl-valeryloxy]-2-hexyl-3-hydroxy-hexa-decanoic 1,3 acid lactone, and the variously substituted N-formylleucine derivatives and stereoisomers thereof (U.S. Pat. No. 4,598,089); tetrahydrolipstatin U.S. Pat. Nos. 5,274,143; 5,420,305; 5,540,917; and 5,643,874; FL-386, 1-[4-(2-methylpropyl)cyclohexyl]-2-[-(phenylsulfonyl)oxy]-ethanone, and the variously substituted sulfonate derivatives related thereto (U.S. Pat. No. 4,452,813); WAY-121898,4-phenoxyphenyl-4-methylpiperidin-1-yl-carboxylate, and the various carbamate esters and pharmaceutically acceptable salts related thereto (U.S. Pat. Nos. 5,512,565; 5,391,571 and 5,602,151); valilactone, and a process for the preparation thereof by the microbial cultivation of Actinomycetes strain MG147-CF2 (Kitahara, et al., J. Antibiotics, 40 (11), 1647-1650 (1987)); esterastin; ebelactone A and ebelactone B, and a process for the preparation thereof by the microbial cultivation of Actinomycetes strain MG7-G1 (Umezawa, et al., J. Antibiotics, 33, 1594-1596 (1980); Japanese Kokal 08-143457, published Jun. 4, 1996). The compound tetrahydrolipstatin is especially preferred. Additional examples include N-3-trifluoromethylphenyl-N′-3-chloro-4′-trifluoromethylphenylurea, and the various urea derivatives related thereto, U.S. Pat. No. 4,405,644; esteracin (U.S. Pat. Nos. 4,189,438 and 4,242,453); and cyclo-O,O′-[(1,6-hexanediyl)-bis-(iminoc-arbonyl)]dioxime, and the various bis(iminocarbonyl)dioximes related thereto (Petersen et al., Liebig's Annalen, 562, 205-229 (1949).

Any bile acid sequestrant known in the art may be used. Examples of suitable bile acid sequestrants include, but are not limited to, Welchol®, Colestid®, LoCholest®, Questran® and fibric acid derivatives, such as Atromid®, Lopid® and Tricor® A compound of the invention can be used in combination with an anti-diabetic compound, i.e. any compound (e.g. insulin) used in the treating diabetes (especially Type II), insulin resistance, impaired glucose tolerance, or the like, or any of the diabetic complications such as neuropathy, nephropathy, retinopathy or cataracts. Additional examples of an anti-diabetic compound include, but are not limited to, a glycogen phosphorylase inhibitor, an aldose reductase inhibitor, a sorbitol dehydrogenase inhibitor, a glucosidase inhibitor, and an amylase inhibitor.

Any glycogen phosphorylase inhibitor known in the art that inhibits the bioconversion of glycogen to glucose-1-phosphate which is catalyzed by the enzyme glycogen phosphorylase may be used. Such glycogen phosphorylase inhibition activity may be readily determined according to standard assays (e.g., J. Med. Chem. 41 (1998) 2934-2938). A variety of glycogen phosphorylase inhibitors are known to those skilled in the art including those described in WO 96/39384 and WO 96/39385. Any aldose reductase inhibitor known in the art that inhibits the bioconversion of glucose to sorbitol catalyzed by the enzyme aldose reductase. Aldose reductase inhibition may be readily determined according to standard assays (e.g., J. Malone, Diabetes, 29:861-864 (1980). “Red Cell Sorbitol, an Indicator of Diabetic Control”).

Any sorbitol dehydrogenase inhibitor known in the art that inhibits the bioconversion of sorbitol to fructose catalyzed by the enzyme sorbitol dehydrogenase may be used. Such sorbitol dehydrogenase inhibitor activity may be readily determined according to standard assays (e.g., Analyt. Biochem (2000) 280: 329-331). Examples of a suitable sorbitol dehydrogenase inhibitor include, but are not limited to, those described in U.S. Pat. Nos. 5,728,704 and 5,866,578.

Any glucosidase inhibitor known in the art that inhibits the enzymatic hydrolysis of complex carbohydrates by glycoside hydrolases, for example amylase or maltase, into bioavailable simple sugars, for example, glucose. Such glucosidase inhibition activity may be readily determined by those skilled in the art according to standard assays (e.g., Biochemistry (1969) δ: 4214).

A generally preferred glucosidase inhibitor includes an amylase inhibitor. Any amylase inhibitor known in the art that inhibits the enzymatic degradation of starch or glycogen into maltose may be used. Such amylase inhibition activity may be readily determined by those skilled in the art according to standard assays (e.g., Methods Enzymol. (1955) 1: 149).

Other preferred glucosidase inhibitors include, but are not limited to, acarbose and the various amino sugar derivatives related thereto (U.S. Pat. Nos. 4,062,950 and 4,174,439); adiposine (U.S. Pat. No. 4,254,256); voglibose, 3,4-dideoxy-4-[[2-hydroxy-1-(hydroxymethyl)ethyl]amino]-2-C-(hydroxymethyl)-D-epi-inositol, and the various N-substituted pseudo-aminosugars related thereto (U.S. Pat. No. 4,701,559); miglitol, (2R,3R,4R,5S)-1-(2-hydroxyethyl)-2-(hydroxymethyl)-3,4,5-piperidinetriol, and the various 3,4,5-trihydroxypiperidines related thereto (U.S. Pat. No. 4,639,436); emiglitate, ethyl p-[2-[(2R,3R,4R,5S)-3,4,5-trihydroxy-2-(hydroxymethyl)piperidino]ethoxy]-benzoate, the various derivatives related thereto and pharmaceutically acceptable acid addition salts thereof (U.S. Pat. No. 5,192,772); MDL-25637, 2,6-dideoxy-7-O-.beta.-D-glucopyrano-syl-2,6-imino-D-glycero-L-gluco-heptitol, the various homodisaccharides related thereto and the pharmaceutically acceptable acid addition salts thereof (U.S. Pat. No. 4,634,765); camiglibose, methyl 6-deoxy-6-[(2R,3R,4R,5S)-3,4,5-trihydroxy-2-(hydroxy-methyl)piperidino]-.alpha.-D-glucopyranoside sesquihydrate, the deoxy-nojirimycin derivatives related thereto, the various pharmaceutically acceptable salts thereof and synthetic methods for the preparation thereof (U.S. Pat. Nos. 5,157,116 and 5,504,078); pradimicin-Q; and salbostatin and the various pseudosaccharides related thereto (U.S. Pat. No. 5,091,524).

Any amylase inhibitor known in the art may be used. Examples include, but are not limited to, tendamistat and the various cyclic peptides related thereto (U.S. Pat. No. 4,451,455); AI-3688 and the various cyclic polypeptides related thereto (U.S. Pat. No. 4,623,714); and trestatin, consisting of a mixture of trestatin A, trestatin B and trestatin C and the various trehalose-containing aminosugars related thereto, (U.S. Pat. No. 4,273,765).

Additional examples of an anti-diabetic compound for use in a combination of the invention include: biguanides (e.g., mefformin), insulin secretagogues (e.g., sulfonylureas and glinides), glitazones, non-glitazone PPAR.gamma. agonists, PPAR.beta. agonists, inhibitors of DPP-IV, inhibitors of PDE5, inhibitors of GSK-3, glucagon antagonists, inhibitors of f-1,6-BPase (Metabasis/Sankyo), GLP-1/analogs (AC 2993, also known as exendin-4), insulin and insulin mimetics (Merck natural products), PKC-beta inhibitors, and AGE breakers.

A compound of the invention can be used in combination with any anti-obesity agent known in the art. Anti-obesity activity may be readily determined according to standard assays known in the art. Examples of suitable anti-obesity agents include, but are not limited to, phenylpropanolamine, ephedrine, pseudoephedrine, phentermine, .beta.sub.3 adrenergic receptor agonists, apolipoprotein-B secretion/microsomal triglyceride transfer protein (apo-B/MTP) inhibitors, MCR-4 agonists, cholecystokinin-A (CCK-A) agonists, monoamine reuptake inhibitors (e.g., sibutramine—U.S. Pat. No. 4,929,629), sympathomimetic agents, serotoninergic agents, cannabinoid receptor antagonists (e.g., rimonabant (SR-141,716A)), dopamine agonists (e.g., bromocriptine —U.S. Pat. Nos. 3,752,814 and 3,752,888), melanocyte-stimulating hormone receptor analogs, 5HT2c agonists, melanin concentrating hormone antagonists, leptin (the OB protein), leptin analogs, leptin receptor agonists, galanin antagonists, lipase inhibitors (e.g., tetrahydrolipstatin, i.e. orlistat), bombesin agonists, anorectic agents (e.g., a bombesin agonist), Neuropeptide-γ antagonists, thyroxine, thyromimetic agents, dehydroepiandrosterones or analogs thereof, glucocorticoid receptor agonists or antagonists, orexin receptor antagonists, urocortin binding protein antagonists, glucagon-like peptide-1 receptor agonists, ciliary neurotrophic factors (e.g., Axokine™), human agouti-related proteins (AGRP), ghrelin receptor antagonists, histamine 3 receptor antagonists or inverse agonists, neuromedin U receptor agonists, and the like.

Any thyromimetic agent known in the art may also be used in combination with a compound of the invention. Thyromimetic activity may be readily determined according to standard assays (e.g., Atherosclerosis (1996) 126: 53-63). Examples of suitable thyromimetic agents include, but are not limited to, those described in U.S. Pat. Nos. 4,766,121; 4,826,876; 4,910,305; 5,061,798; 5,284,971; 5,401,772; 5,654,468; and 5,569,674.

A compound of the invention may further be used in combination with an anti-resorptive agent (e.g., progestins, polyphosphonates, bisphosphonate(s), estrogen agonists/antagonists, estrogen, estrogen/progestin combinations, Premarin®, estrone, estriol or 17.alpha.- or 17.beta.-ethynyl estradiol). Exemplary progestins are available from commercial sources and include, but are not limited to: algestone acetophenide, altrenogest, amadinone acetate, anagestone acetate, chlormadinone acetate, cingestol, clogestone acetate, clomegestone acetate, delmadinone acetate, desogestrel, dimethisterone, dydrogesterone, ethynerone, ethynodiol diacetate, etonogestrel, fluorogestone acetate, gestaclone, gestodene, gestonorone caproate, gestrinone, haloprogesterone, hydroxyprogesterone caproate, levonorgestrel, lynestrenol, medrogestone, medroxyprogesterone acetate, melengestrol acetate, methynodiol diacetate, norethindrone, norethindrone acetate, norethynodrel, norgestimate, norgestomet, norgestrel, Ogestone phenpropionate, progesterone, quingestanol acetate, quingestrone, and tigestol. Preferred progestins are medroxyprogestrone, norethindrone and norethynodrel.

Exemplary bone resorption inhibiting polyphosphonates include polyphosphonates of the type described in U.S. Pat. No. 3,683,080. Preferred polyphosphonates are geminal diphosphonates (also referred to as bis-phosphonates), 6-amino-1-hydroxy-hexylidene-bisphosphonic acid and 1-hydroxy-3(methylpentylamino)-propylidene-bisphosphonic acid. Tiludronate disodium, ibandronic acid, alendronate, resindronate, and zoledronic acid are each especially preferred polyphosphonates. The polyphosphonates may be administered in the form of the acid, or of a soluble alkali metal salt or alkaline earth metal salt. Hydrolyzable esters of the polyphosphonates are likewise included. Specific examples include, but are not limited to, ethane-1-hydroxy 1,1-diphosphonic acid, methane diphosphonic acid, pentane-1-hydroxy-1,1-diphosphonic acid, methane dichloro diphosphonic acid, methane hydroxy diphosphonic acid, ethane-1-amino-1,1-diphosphonic acid, ethane-2-amino-1,1-diphosphonic acid, propane-3-amino-1-hydroxy-1,1-diphosphonic acid, propane-N,N-dimethyl-3-amino-1-hydroxy-1,1-diphosphonic acid, propane-3,3-dimethyl-3-amino-1-hydroxy-1,1-diphosphonic acid, phenyl amino methane diphosphonic acid, N,N-dimethylamino methane diphosphonic acid, N(2-hydroxyethyl)amino methane diphosphonic acid, butane-4-amino-1-hydroxy-1,1-diphosphonic acid, pentane-5-amino-1-hydroxy-1-1,1-diphosphonic acid, hexane-6-amino-1-hydroxy-1,1-diphosphonic acid and pharmaceutically acceptable esters and salts, thereof.

Any estrogen agonist/antagonist known in the art which bind with the estrogen receptor, inhibit bone turnover and/or prevent bone loss may be used in a combination of, the invention. More specifically, an estrogen agonist may be any chemical compound capable of binding to the estrogen receptor sites in mammalian tissue, and mimicking the actions of estrogen in one or more tissue. An estrogen antagonist may be any chemical compound capable of binding to the estrogen receptor sites in mammalian tissue, and blocking the actions of estrogen in one or more tissues. Such activities may be readily determined according to standard assays, including estrogen receptor binding assays, and standard bone histomorphometric and densitometer methods (Eriksen E. F. et al., Bone Histomorphometry, Raven Press, New York, 1994, pages 1-74; Grier S. J. et. al., “The Use of Dual-Energy X-Ray Absorptiometry In Animals”, Inv. Radiol., 1996, 31 (1):50-62; Wahner H. W. and Fogelman I., The Evaluation of Osteoporosis: Dual Energy X-Ray Absorptiometry in Clinical Practice., Martin Dunitz Ltd., London 1994, pages 1-296). Examples of a suitable estrogen agonist/antagonist is 3-(4-(1,2-diphenyl-but-1-enyl)-phenyl)-acrylic acid (see Willson et al., Endocrinology, 1997, 138, 3901-3911); tamoxifen (ethanamine, 2-(−4-(1,2-diphenyl-1-butenyl)phenoxy)-N,N-dimethyl, (Z)-2-, 2-hydroxy-1,2,3-propanetricarboxylate (1:1)) and related compounds (U.S. Pat. No. 4,536,516); 4-hydroxy tamoxifen (U.S. Pat. No. 4,623,660); raloxifene (methanone, (6-hydroxy-2-(4-hydroxyphenyl)benzo[b]thien-3-yl)(4-(2-(1-piperidinyl)ethoxy)phenyl)-hydrochloride)(U.S. Pat. No. 4,418,068); toremifene (ethanamine, 2-(4-(4-chloro-1,2-diphenyl-1-butenyl)phenoxy)-N,N-dimethyl-, (Z)-, 2-hydroxy-1,2,3-propanetricarboxylate (1:1) (U.S. Pat. No. 4,996,225); centchroman (1-(2-((4-(-methoxy-2,2, dimethyl-3-phenyl-chroman-4-yl)-phenoxy)-ethyl)-p-pyrrolidine)(U.S. Pat. No. 3,822,287); levormeloxifene; idoxifene ((E)-1-(2-(4-(1-(4-iodo-phenyl)-2-phenyl-but-1-enyl)-phenoxy)-ethyl)-pyrrolidinone (U.S. Pat. No. 4,839,155); 2-(4-methoxy-phenyl)-3-[4-(2-piperidin-1-yl-ethoxy)-phenoxy]-benzo[b]thio-phen-6-ol (U.S. Pat. No. 5,488,058); 6-(4-hydroxy-phenyl)-5-(4-(2-piperidin-1-yl-ethoxy)-benzyl)-naphthalen-2-ol (U.S. Pat. No. 5,484,795); (4-(2-(2-aza-bicyclo[2.2.1]hept-2-yl)-ethoxy)-phenyl)-(6-hydroxy-2-(4-hyd-roxy-phenyl)-benzo[b]thiophen-3-yl)-methanone (WO 95/10513 assigned to Pfizer Inc.); TSE-424 (Wyeth-Ayerst Laboratories); arazoxifene; derivatives of 2-phenyl-3-aroyl-benzoth-iophene and 2-phenyl-3-aroylbenzothiophene-1-oxide (U.S. Pat. No. 4,133,814); estrogen agonist/antagonists described in U.S. Pat. No. 4,133,814; and estrogen agonist/antagonists described in commonly assigned U.S. Pat. No. 5,552,412.

Especially preferred estrogen agonist/antagonists described in U.S. Pat. No. 5,552,412 are: cis-6-(4-fluoro-phenyl)-5-(4-(2-piperidin-1-yl-ethoxy)-phenyl)-5,6,-7,8-tetrahydro-naphthalene-2-ol; (−)-cis-6-phenyl-5-(4-(2-pyrrolidin-1-yl-ethoxy)-phenyl)-5,6,7,8-te-trahydro-naphthalene-2-ol (also known as lasofoxifene); cis-6-phenyl-5-(4-(2-pyrrolidin-1-yl-ethoxy)-phenyl)-5,6,7,8-tetrah-ydro-naphthalene-2-ol; cis-1-(6′-pyrrolodinoethoxy-3′-pyridyl)-2-phenyl-6-hydroxy-1,2,3,4-tetrahydronaphthalene; 1-(4′-pyrrolidinoethoxyphenyl)-2-(4″-fluorophenyl)-6-hydroxy-1,2,3,-4-tetrahydroisoquinoline; cis-6-(4-hydroxyphenyl)-5-(4-(2-piperidin-1-yl-ethoxy)-phenyl)-5,6,-7,8-tetrahydro-naphthalene-2-ol; and 1-(4′-pyrrolidinolethoxyphenyl)-2-phenyl-6-hydroxy-1,2,3,4-tetrahyd-roisoquinoline.

Any anti-osteoporosis agent known in the art may be used in a combination of the invention. Examples include, but are not limited to, parathyroid hormone (PTH) (a bone anabolic agent); parathyroid hormone (PTH) secretagogues (see, e.g., U.S. Pat. No. 6,132,774), particularly calcium receptor antagonists; calcitonin; and vitamin D and vitamin D analogs.

Any antihypertensive agent known in the art may be used in a combination of the invention. Antihypertensive activity may be determined according to standard tests (e.g. blood pressure measurements). Examples of suitable antihypertensive agents include, but are not limited to, (a) amlodipine and related dihydropyridine compounds (U.S. Pat. Nos. 4,572,909 and 5,155,120) such as, but not limited to, amlodipine benzenesulfonate salt (also termed amlodipine besylate (Norvasc))(U.S. Pat. No. 4,879,303) and other pharmaceutically acceptable acid addition salts of amlodipine (U.S. Pat. No. 5,155,120); (b) calcium channel blockers such as, but not limited to, bepridil (U.S. Pat. No. 3,962,238 or U.S. Reissue No. 30,577), clentiazem (U.S. Pat. No. 4,567,175), diltiazem (U.S. Pat. No. 3,562), fendiline (U.S. Pat. No. 3,262,977), gallopamil (U.S. Pat. No. 3,261,859); mibefradil, prenylamine, semotiadil, terodiline, verapamil, aranipine, barnidipine, benidipine, cilnidipine, efonidipine, elgodipine, felodipine, isradipine, lacidipine, lercanidipine, manidipine, nicardipine, nifedipine, nilvadipine, nimodipine, nisoldipine, nitrendipine, cinnarizine, flunarizine, lidoflazine, lomerizine, bencyclane, etafenone, and perhexyline; (c) angiotensin converting enzyme inhibitors (“ACE-inhibitors”) such as, but not limited to, alacepril (U.S. Pat. No. 4,248,883), benazepril (U.S. Pat. No. 4,410,520), captopril, ceronapril, delapril, enalapril, fosinopril, imadapril, lisinopril, moveltopril, perindopril, quinapril, ramipril, spirapril, temocapril, and trandolapril; (d) angiotensin-II receptor antagonists such as, but not limited to, candesartan (U.S. Pat. No. 5,196,444), eprosartan (U.S. Pat. No. 5,185,351), irbesartan, losartan, and valsartan; (e) beta-adrenergic receptor blockers (beta- or β-blockers) such as, but not limited to, acebutolol (U.S. Pat. No. 3,857,952), alprenolol, amosulalol (U.S. Pat. No. 4,217,305), arotinolol, atenolol, befunolol, betaxolol; and (f) alpha-adrenergic receptor blockers (alpha- or α-blockers) such as, but not limited to, amosulalol (U.S. Pat. No. 4,217,307), arotinolol (U.S. Pat. No. 3,932,400), dapiprazole, doxazosin, fenspiride, indoramin, labetolol, naftopidil, nicergoline, prazosin, tamsulosin, tolazoline, trimazosin, and yohimbine, which may be isolated from natural sources according to methods well known to those skilled in the art.

Any HMGCOA reductase inhibitor agent known in the art may be used in a combination of the invention. HMGCoA reductase activity may be determined according to standard tests (e.g. blood plasma low density lipoprotein cholesterol (LDL-C) measurements). Examples of suitable HMGCoA reductase inhibitor agents include, but are not limited to, atorvastatin, simvastatin, fluvastatin, lovastatin, pravastatin, rosuvastatin, simvastatin, and cerivastatin. A number of patents have issued disclosing atorvastatin and include: U.S. Pat. Nos. 4,681,893, 5,273,995 and 5,969,156. A number of patents have issued disclosing rosuvastatin and include: U.S. Pat. Nos. 5,260,440 (RE37314), 6,858,618, and 6,894,058. A number of patents have issued disclosing cerivastatin and include: U.S. Pat. Nos. 5,006,530, 5,169,857, and 5,401,746. A number of patents have issued disclosing fluvastatin and include: U.S. Pat. Nos. 4,739,073 and 5,354,772. A number of patents have issued disclosing lovastatin and include: U.S. Pat. Nos. 4,231,938, 4,294,926, and 4,319,039. A number of patents have issued disclosing pravastatin and include: U.S. Pat. Nos. 4,346,227, 4,410,629, and 4,448,979.

The present invention contains compounds that can be synthesized in a number of ways familiar to one skilled in organic synthesis. The following non-limiting reaction schemes illustrate the preparation of the compounds of the present invention. Unless otherwise indicated, all variables in the reaction schemes and the discussions that follow are as defined above. As would be understood by one of skill in the art, individual compounds may require manipulation of the conditions in order to accommodate various functional groups. A variety of protecting groups known to one skilled in the art may be required. Purification, if necessary, may be accomplished on a silica gel column eluted with the appropriate organic solvent system. Also, reverse phase HPLC or recrystallization may be employed.

### Preparation of Invention Compounds

Scheme 1 describes a general synthetic scheme for the preparation of Example 7 and Example 8, as representative, non-limiting illustrations of the present invention.

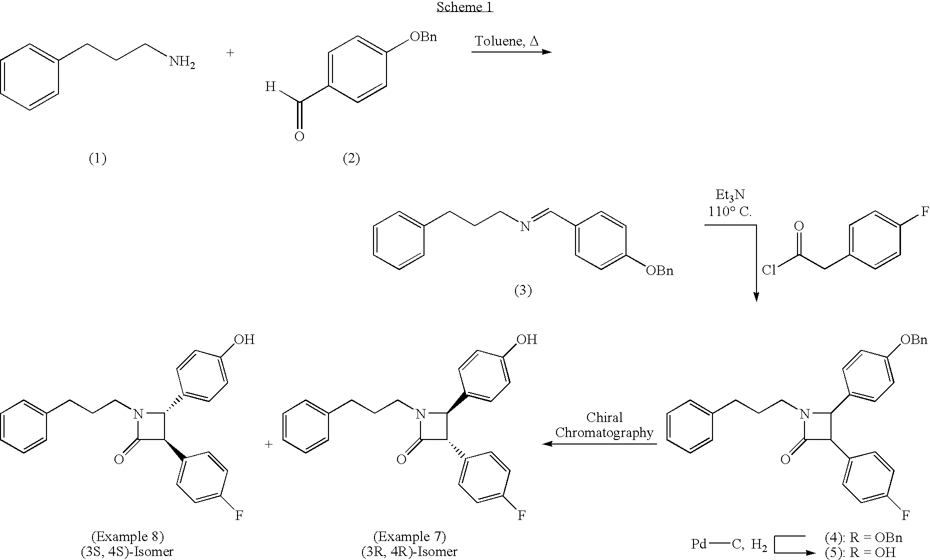

As shown in Scheme 1, imine (3) was prepared by condensation of phenylpropylamine (1) and 4-benzyloxybenzaldehyde (2). This imine (3) was then engaged in a thermal [2+2]cyclo-addition reaction with a ketene component generated in-situ via dehydrohalogenation of 4-fluorophenylacetyl chloride. This cycloaddition reaction afforded β-lactam product (4) as a racemic mixture of trans-stereoisomers. The benzyl protecting group of β-lactam (4) was then removed by hydrogenolysis to give compound (5) as a racemic mixture of trans-stereoisomers. Finally, the enantiomers of racemic (5) were separated by chiral chromatography to afford Example 7 and Example 8 as illustrated.

Further examples of this invention can be prepared using the methods of Scheme 2 or variations thereof that would be evident to those skilled in the art.

Scheme 2 describes a general synthetic scheme for the preparation of Example 13 and Example 15, as representative, non-limiting illustrations of the present invention.

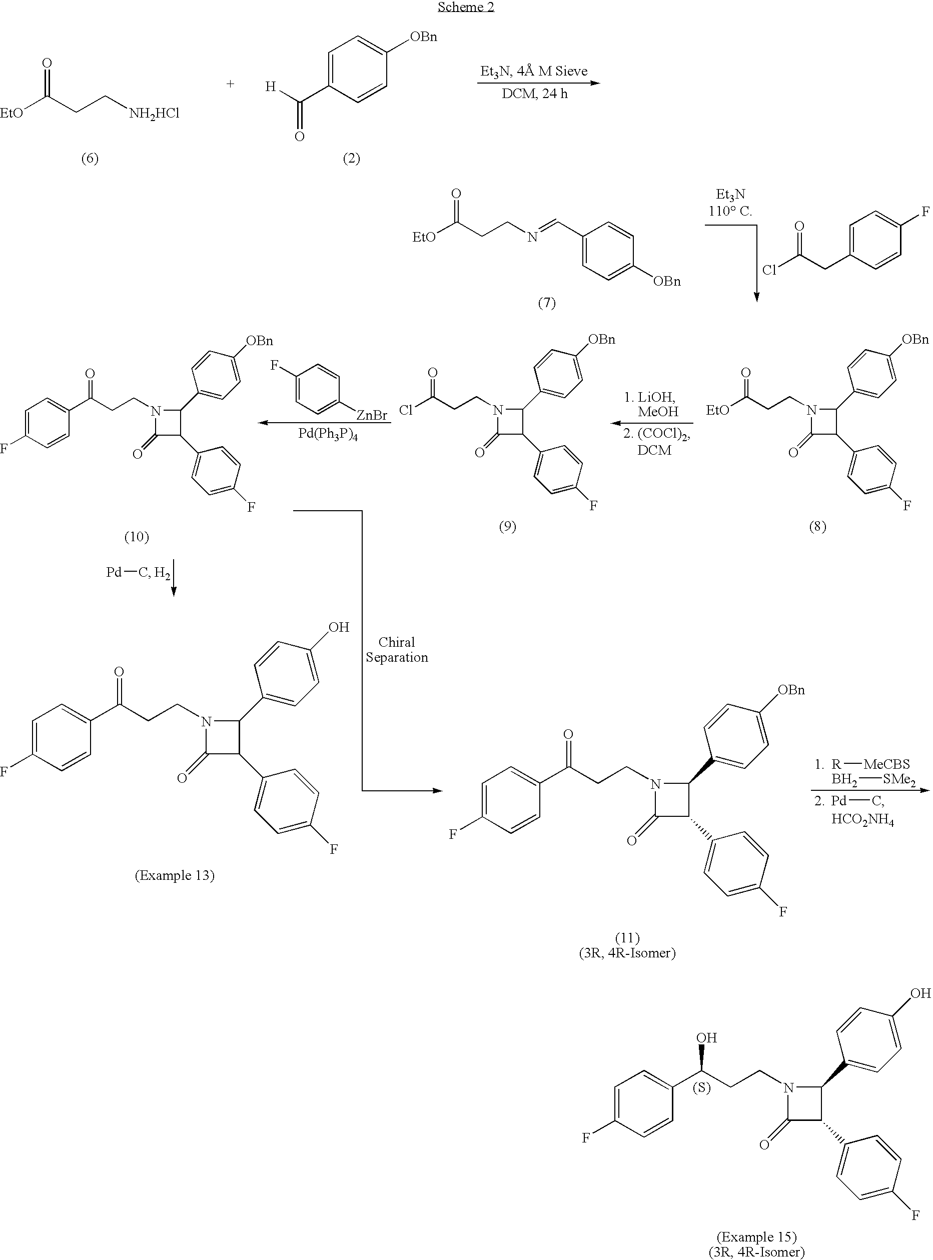

As shown in Scheme 2, imine (7) was prepared by condensation of β-alanine ethyl ester hydrochloride (6) and 4-benzyloxybenzaldehyde (2). This imine was then engaged in a thermal [2+2]cycloaddition reaction with a ketene component generated in-situ via dehydrohalogenation of 4-fluorophenylacetyl chloride. This cycloaddition reaction afforded β-lactam (8) as a racemic mixture of trans-stereoisomers. The ester of lactam (8) was subsequently converted to acid chloride (9) via the intermediacy of a carboxylic acid. Formation of aryl ketone (10) from acid chloride (9) was accomplished using a palladium-mediated coupling with 4-fluorophenyl zinc bromide. Hydrogenolysis of a portion of intermediate (10) provided racemic mixture of trans-stereoisomers (Example 13) while the remainder of compound (10) was resolved into its constituent enantiomers via a chiral HPLC. Enantiopure compound (II), 3R,4R-isomer, was then subjected to an asymmetric reduction with (R)-MeCBS and BH₃.SMe₂ followed by hydrogenolysis give compound Example 15. The opposite enantiomer of compound II (i.e. 3S,4S-isomer) was processed in the same manner to provide Example 17 (not shown in scheme). Reagent acronyms are as follows: dichloromethane (DCM) and (R)-2-methyl-CBS-oxazaborolidine ((R)-MeCBS).

Scheme 3 describes a general synthetic scheme for the preparation of compounds of formulas 16 and 17, as representative, non-limiting illustrations of the present invention. Scheme 4 further describes a general synthetic scheme for the preparation of compounds of formulas 23, 24, and 25, as per the synthetic scheme presented.

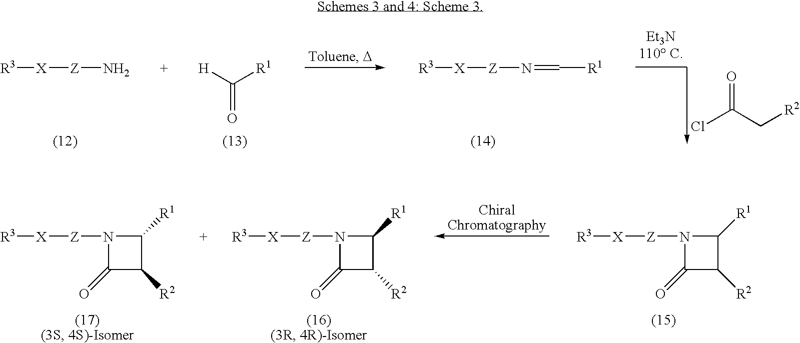

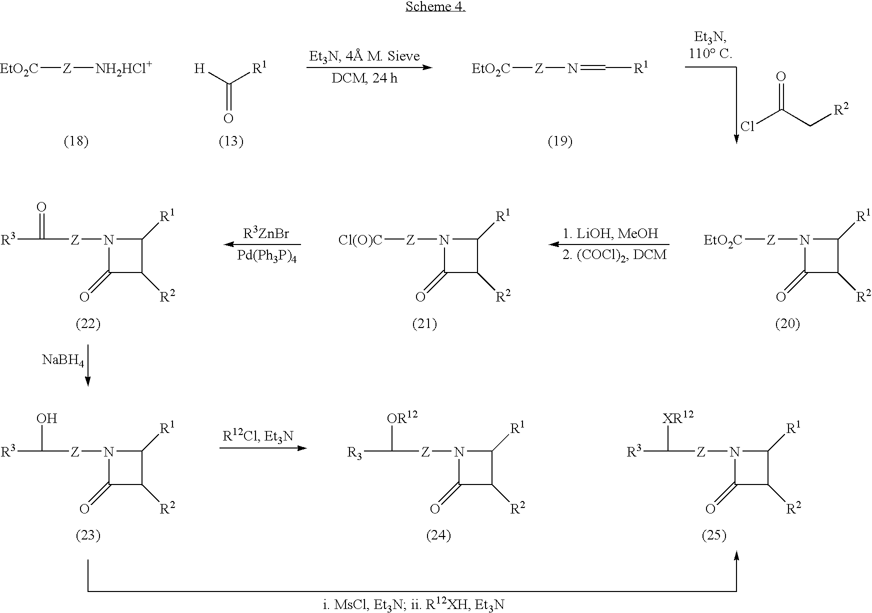

As shown in Scheme 3, imine (14) is prepared by condensation of amine (12) and substituted benzaldehyde (13). This imine (14) is then engaged in a thermal [2+2]cyclo-addition reaction with a ketene component generated in-situ via dehydrohalogenation of a substituted phenylacetyl chloride. This cycloaddition reaction affords β-lactam product (15) as a racemic mixture of trans-stereoisomers. Finally, the enantiomers of racemic (15) are separated by chiral chromatography to afford (16) and (17) as illustrated.

As shown in Scheme 4, imine (19) is prepared by condensation of amine (18) and substituted benzaldehyde (13). This imine is then engaged in a thermal [2+2]cycloaddition reaction with a ketene component generated in-situ via dehydrohalogenation of a substituted phenylacetyl chloride. This cycloaddition reaction affords β-lactam (20) as a racemic mixture of trans-stereoisomers. The ester of lactam (20) is subsequently converted to acid chloride (21) via the intermediacy of a carboxylic acid. Formation of aryl ketone (22) from acid chloride (21) is accomplished using a palladium-mediated coupling with a substituted phenyl zinc bromide. Reduction of ketone (22) affords alcohol (23) which can then be derivatived to compound (24). Alternatively, alcohol (23) can be converted to a mesylate and displaced with a nucleophile (R¹²XH) in the presence of a suitable base to give compound (25).

Substituents within Scheme 3 and Scheme 4 that are designated as R¹, R², R³, R¹², X, and Z are as previously defined. Reagent acronyms are as follows: dichloromethane (DCM) and mesyl chloride (MeCl).

### EXAMPLE 1

### (3R,4R)-bis-(4-methoxy-phenyl)-1-(3-phenyl-propyl)-azetidin-2-one

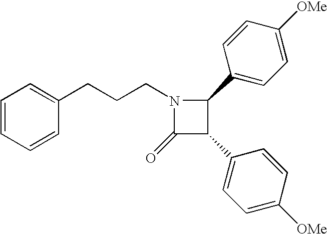

### Step A

### (4-Methoxy-benzylidene)-(3-phenyl-propyl)-amine

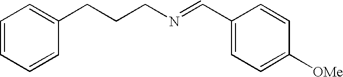

To a solution of p-anisaldehyde (7.83 g, 57.5 mmol) in toluene (150 mL) was added 3-phenyl propyl amine (7.78 g, 57.5 mmol) and the reaction mixture was heated to 110° C. for 3 hrs with Dean-Stark apparatus in place to azeotropically remove water. After cooling to 25° C., the solvent was removed under reduced pressure to provide (4-methoxy-benzylidene)-(3-phenyl-propyl)-amine (14.7 g, 100%) as a light yellow oil that was used without further purification. H-NMR (CDCl₃) δ 8.16 (s, 1H), 7.66 (d, 2H), 7.27-7.11 (m, 5H), 6.89 (d, 2H), 3.81 (s, 3H), 3.57 (t, 2H), 2.66 (t, 2H), 2.04-1.97 (m, 2H).

### Step B

### (3R,4R)-bis-(4-methoxy-phenyl)-1-(3-phenyl-propyl)-azetidin-2-one

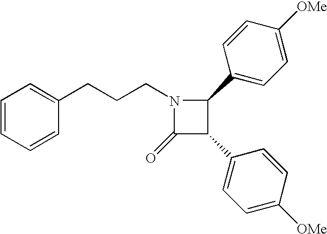

A solution of (4-methoxy-benzylidene)-(3-phenyl-propyl)-amine (14.7 g, 57.9 mmol) and 4-fluorophenylacetyl chloride (10.7 g, 57.9 mmol) in toluene (100 mL) was heated to 110° C. and Et₃N (7.0 g, 69.5 mmol) was slowly added while maintaining vigorous stirring. Once the addition was complete, the reaction was stirred at 110° C. for 12 hrs and then cooled to 25° C. The solids were removed by filtration, and the filtrate was concentrated to a yellow oil that was purified by silica gel chromatography (3→20% EtOAc/hexane) to afford racemic 3,4-bis-(4-methoxy-phenyl)-1-(3-phenyl-propyl)-azetidin-2-one (4.16 g, 18%). Separation of this racemic product by preparative chiral HPLC provided: (3R,4R)-bis-(4-methoxy-phenyl)-1-(3-phenyl-propyl)-azetidin-2-one: H-NMR (CDCl₃) δ 7.26-7.08 (m, 9H), 6.91 (d, 2H), 6.85 (d, 2H), 4.35 (d, 1H), 4.04 (d 1H), 3.79 (s, 2H), 3.76 (s, 3H), 3.59-3.51 (m, 1H), 2.92-2.85 (m, 1H), 2.59-2.57 (m, 2H), 1.83-1.76 (m, 1) MS (APCI⁺): m/z 402.1 (M+H) and the compound of Example 2 following.

### EXAMPLE 2

### (3S,4S)-bis-(4-methoxy-phenyl)-1-(3-phenyl-propyl)-azetidin-2-one

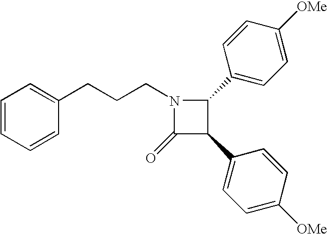

See procedure of Example 1. H-NMR (CDCl₃) δ 7.26-7.08 (m, 9H), 6.91 (d, 2H), 6.85 (d, 2H), 4.35 (d, 1H), 4.04 (d 1H), 3.79 (s, 2H), 3.76 (s, 3H), 3.59-3.51 (m, 1H), 2.92-2.85 (m, 1H), 2.59-2.57 (m, 2H), 1.83-1.76 (m, 1H); MS (APCI⁺): m/z 402.1 (M+H).

### EXAMPLE 3

### 3R-(4-Fluoro-phenyl)-4R-(4-methoxy-phenyl)-1-(3-phenyl-propyl)-azetidin-2-one

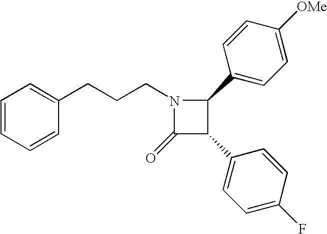

Prepared in a manner analogous to the method of Example 1. H-NMR (CDCl₃) δ 7.23-6.89 (m, 13H), 4.35 (d, 1H), 4.07 (d, 1H), 3.80 (s, 3H), 3.68-3.50 (m, 1H), 2.93-2.86 (m, 1H), 2.63-2.53 (m, 2H), 1.84-1.76 (m, 2H); MS (APCI⁺): m/z 390.1 (M+H).

### EXAMPLE 4

### 4-(4-Benzyloxy-phenyl)-3-(4-fluoro-phenyl)-1-phenethyl-azetidin-2-one

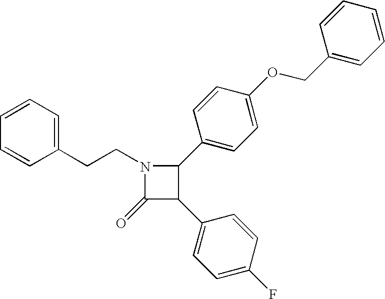

### Step A

### (4-Benzyloxy-benzylidene)-phenethyl-amine

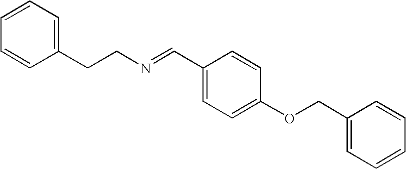

To a solution of 4-benzyloxybenzaldehyde (14.0 g, 66.0 mmol) in toluene (300 mL) was added phenethylamine (8.0 g, 66.0 mmol) and the reaction mixture was heated to 110° C. for 3 hrs with Dean-Stark apparatus in place to azeotropically remove water. After cooling to 25° C., the solvent was removed under reduced pressure to provide (4-benzyloxy-benzylidene)-phenethyl-amine (20.8 g, 100%) that was used without further purification.

### Step B

### 4-(4-Benzyloxy-phenyl)-3-(4-fluoro-phenyl)-1-phenethyl-azetidin-2-one

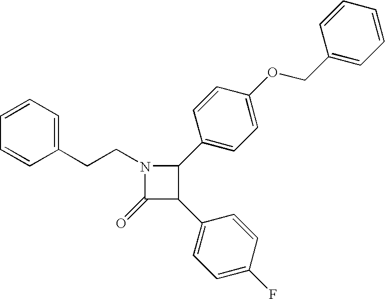

A solution of (4-benzyloxy-benzylidene)-phenethyl-amine (20.8 g, 66.0 mmol) and 4-fluorophenylacetyl chloride (11.4 g, 66.0 mmol) in toluene (100 mL) was heated to 110° C. and Et₃N (8.0 g, 79.1 mmol) was slowly added while maintaining vigorous stirring. Once the addition was complete, the reaction was stirred at 110° C. for 16 hrs and then cooled to 25° C. The solids were removed by filtration, and the filtrate was concentrated to a yellow oil that was purified by silica gel chromatography (3→25% EtOAc/hexane) to afford racemic 4-(4-benzyloxy-phenyl)-3-(4-fluoro-phenyl)-1-phenethyl-azetidin-2-one (15.5 g, 52%). H-NMR (CDCl₃) δ 7.42-7.10 (m, 12H), 6.97-6.89 (m, 6H), 5.05 (s, 2H), 4.08 (d, 1H), 3.95 (d, 1H), 3.95-3.90 (m, 1H), 3.09-3.02 (m, 1H), 2.89-2.86 (m, 1H), 2.80-2.76 (m, 1H); MS (APCI⁺): m/z 452.2 (M+H).

### EXAMPLE 5

### 3R-(4-Fluoro-phenyl)-4R-(4-hydroxy-phenyl)-1-phenethyl-azetidin-2-one

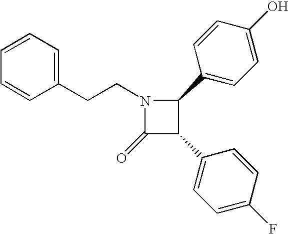

A flask containing 4-(4-benzyloxy-phenyl)-3-(4-fluoro-phenyl)-1-phenethyl-azetidin-2-one (from Example 4) (15.0 g, 33.2 mmol) in MeOH (500 mL) was evacuated and purged with nitrogen. Subsequently, 10% Pd—C (1.0 g) was added and the reaction vessel was charged with hydrogen (via balloon) and was stirred at 25° C. for 16 hrs. Once the reaction was complete as determined by TLC, the reaction vessel was purged with nitrogen and the contents were filtered through a celite pad. The filtrate was concentrated and crude product was purified by silica gel chromatography (5→25% EtOAc/hexane) to afford racemic 3-(4-fluoro-phenyl)-4-(4-hydroxy-phenyl)-1-phenethyl-azetidin-2-one. Separation of this racemic product by preparative chiral HPLC provided: 3R-(4-fluoro-phenyl)-4R-(4-hydroxy-phenyl)-1-phenethyl-azetidin-2-one H-NMR (CDCl₃) δ 7.28-6.79 (m, 13H), 5.75 (bs, 1H), 4.07 (d, 1H), 3.95 (d, 1H), 3.94-3.88 (m, 1H), 3.09-3.02 (m, 1H), 2.92-2.85 (m, 1H), 2.81-2.74 (m, 1H); MS (APCI⁺): m/z 362.1 (M+H) and the compound of Example 6 following.

### EXAMPLE 6

### 3S-(4-Fluoro-phenyl)-4S-(4-hydroxy-phenyl)-1-phenethyl-azetidin-2-one

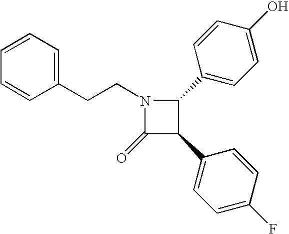

Prepared according to the method of Example 5. ¹H-NMR (CDCl₃) δ 7.28-6.79 (m, 13H), 6.05 (bs, 1H), 4.07 (d, 1H), 3.95 (d, 1H), 3.94-3.88 (m, 1H), 3.09-3.02 (m, 1H), 2.92-2.85 (m, 1H), 2.81-2.74 (m, 1H); MS (APCI⁺): m/z 362.1 (M+H).

### EXAMPLE 7

### 3R-(4-Fluoro-phenyl)-4R-(4-hydroxy-phenyl)-1-(3-phenyl-propyl)-azetidin-2-one

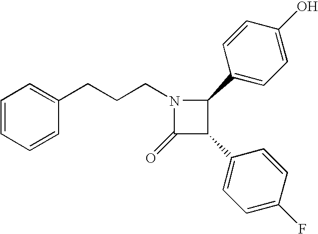

Prepared in a manner analogous to the method of Example 5. H-NMR (CDCl₃) δ 7.24-6.97 (m, 11H), 6.85-6.82 (d, 2H), 5.21 (bs, 1H), 4.34 (d, 1H), 4.06 (d, 1H), 3.57-3.52 (m, 1H), 2.92-2.85 (m, 1H), 2.61-2.56 (m, 2H), 1.83-1.80 (m, 2H); MS (APCI⁺): m/z 376.1 (M+H).

### EXAMPLE 8

### 3S-(4-Fluoro-phenyl)-4S-(4-hydroxy-phenyl)-1-(3-phenyl-propyl)-azetidin-2-one

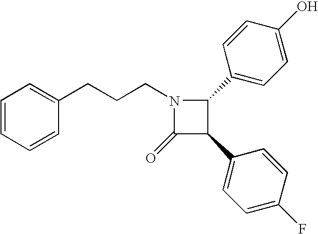

Prepared in a manner analogous to the method of Example 5. H-NMR (CDCl₃) δ 7.24-6.97 (m, 11H), 6.85-6.82 (d, 2H), 5.30 (bs, 1H), 4.34 (d, 1H), 4.06 (d, 1H), 3.57-3.52 (m, 1H), 2.92-2.85 (m, 1H), 2.61-2.56 (m, 2H), 1.83-1.80 (m, 2H); MS (APCI⁺): m/z 376.1 (M+H).

### EXAMPLE 9

### 3R-(4-Fluoro-phenyl)-4R-(4-hydroxy-phenyl)-1-(4-phenyl-butyl)-azetidin-2-one

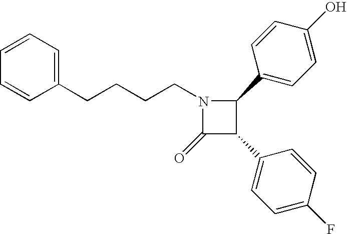

Prepared in a manner analogous to the method of Example 5. H-NMR (CDCl₃) δ 7.23-6.96 (m, 11H), 6.84-6.80 (d, 2H), 5.65 (bs, 1H), 4.25 (d, 1H), 4.04 (d, 1H), 3.56-3.51 (m, 1H), 2.88-2.81 (m, 1H), 2.60-2.50 (m, 2H), 1.64-1.57 (m, 2H), 1.52-1.46 (m, 2H); MS (APCI⁺): m/z 390.0 (M+H).

### EXAMPLE 10

### 3S-(4-Fluoro-phenyl)-4S-(4-hydroxy-phenyl)-1-(4-phenyl-butyl)-azetidin-2-one

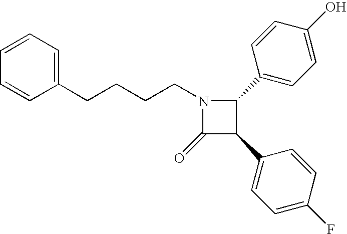

Prepared in a manner analogous to the method of Example 5. H-NMR (CDCl₃) δ 7.23-6.96 (m, 11H), 6.84-6.80 (d, 2H), 5.26 (bs, 1H), 4.25 (d, 1H), 4.04 (d, 1H), 3.56-3.51 (m, 1H), 2.88-2.81 (m, 1H), 2.60-2.50 (m, 2H), 1.64-1.57 (m, 2H), 1.52-1.46 (m, 2H); MS (APCI⁺): m/z 390.0 (M+H).

### EXAMPLE 11

### 4-(4-Benzyloxy-phenyl)-3-(4-fluoro-phenyl)-1-(4-phenyl-butyl)-azetidin-2-one

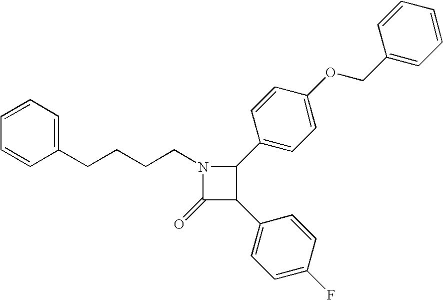

Prepared in a manner analogous to the method of Example 4. H-NMR (CDCl₃) δ 7.42-6.96 (m, 18H), 5.05 (s, 2H), 4.26 (d, 1H), 4.04 (d, 1H), 3.54-3.51 (m, 1H)m 2.87-2.83 (m, 1H), 2.59-2.52 (m, 2H), 1.63-1.47 (m, 4H); MS (APCI⁺): m/z 480.1 (M+H).

### EXAMPLE 12

### 4R-(4-Benzyloxy-phenyl)-3R-(4-fluoro-phenyl)-1-[3-(4-fluoro-phenyl)-3-O-propyl]-azetidin-2-one

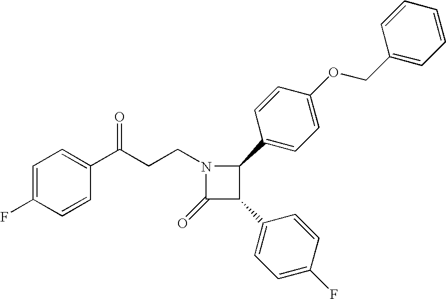

### Step A

### 3-[(4-Benzyloxy-benzylidene)-amino]-propionic acid ethyl ester

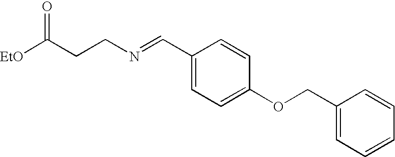

A solution of β-alanine ethyl ester hydrochloride (20.7 g, 135 mmol), Et₃N (20.5 g, 202 mmol) and 4 Å molecular sieves (40 g) in CH₂Cl₂ (300 mL) was stirred at 0° C. while 4-benzyloxybenzaldehyde (28.6 g, 135 mmol) was added as a solid over 5 min. The reaction mixture was then warmed to 25° C. and stirred at that temperature for 5 hrs. TLC analysis indicated that reaction was not yet; consequently, MgSO₄ (10 g) was added and the reaction was stirred for an additional 12 hrs at 25° C. after which time reaction was complete as determined by TLC analysis. The reaction mixture was then filtered to remove solids, and the filtrate was concentrated to provide 3-[(4-benzyloxy-benzylidene)-amino]-propionic acid ethyl ester (37.3, 89%) as a pale yellow solid of sufficient purity for use in the next step without additional purification; MS (APCI⁺): m/z 312.1 (M+H).

### Step B

### 3-[2-(4-Benzyloxy-phenyl)-3-(4-fluoro-phenyl)-4-O-azetidin-1-yl]-propionic acid ethyl ester

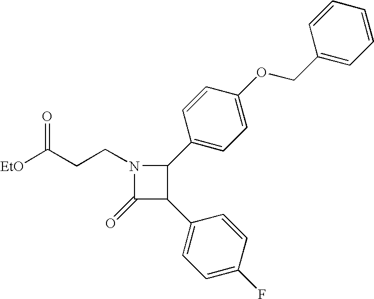

A solution of 3-[(4-benzyloxy-benzylidene)-amino]-propionic acid ethyl ester (37.3 g, 120 mmol) and 4-fluorophenylacetyl chloride (20.7 g, 120 mmol) in toluene (400 mL) was heated to 110° C. and Et₃N (14.5 g, 144 mmol) was slowly added while maintaining vigorous stirring. Once the addition was complete, the reaction was stirred at 110° C. for 12 hrs and then cooled to 25° C. The solids were removed by filtration, and the filtrate was concentrated to a yellow oil that was purified by silica gel chromatography (10→30% EtOAc/hexane) to afford racemic 3-[2-(4-benzyloxy-phenyl)-3-(4-fluoro-phenyl)-4-O-azetidin-1-yl]-propionic acid ethyl ester (26.5 g, 49%); MS (APCI⁺): m/z 448.2 (M+H).

### Step C

### 3-[2-(4-Benzyloxy-phenyl)-3-(4-fluoro-phenyl)-4-O-azetidin-1-yl]-propionic acid

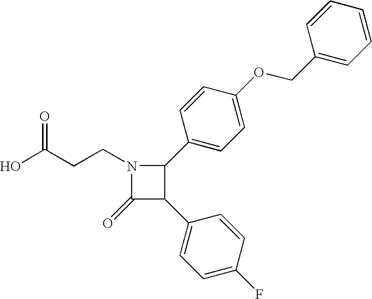

To a solution of 3-[2-(4-benzyloxy-phenyl)-3-(4-fluoro-phenyl)-4-O-azetidin-1-yl]-propionic acid ethyl ester (7.36 g, 16.4) in MeOH (100 ml) at RT was added LiOH (0.59 g, 24.7 mmol) at 25° C. The reaction mixture was stirred at 25° C. for 4 hrs after which time the solvent was removed under reduced pressure. Et₂O and water were then added, and the organic layer was separated and discarded. The aqueous layer was treated with 1 N HCl to until it reached pH 2 and EtOAc was added. The organic layer was separated, washed with brine, dried (Na₂SO₄) and concentrated to afford 3-[2-(4-benzyloxy-phenyl)-3-(4-fluoro-phenyl)-4-O-azetidin-1-yl]-propionic acid (6.14 g, 89%); MS (APCI⁺): m/z 420.0 (M+H).

### Step D

### 3-[2-(4-Benzyloxy-phenyl)-3-(4-fluoro-phenyl)-4-O-azetidin-1-yl]-propionyl chloride

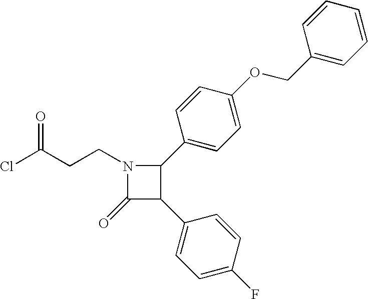

To a solution of 3-[2-(4-benzyloxy-phenyl)-3-(4-fluoro-phenyl)-4-O-azetidin-1-yl]-propionic acid (6.14, 14.7 mmol) in CH₂Cl₂ (150 mL) at 25° C. was added oxalyl chloride (2.8 g, 22.1 mmol). The reaction was stirred at 25° C. for 16 hrs and then concentrated under reduced pressure to afford 3-[2-(4-benzyloxy-phenyl)-3-(4-fluoro-phenyl)-4-O-azetidin-1-yl]-propionyl chloride (6.44 g, 100%) in sufficient purity for use in the next step.

### Step E

### 4R-(4-Benzyloxy-phenyl)-3R-(4-fluoro-phenyl)-1-[3-(4-fluoro-phenyl)-3-O-propyl]-azetidin-2-one

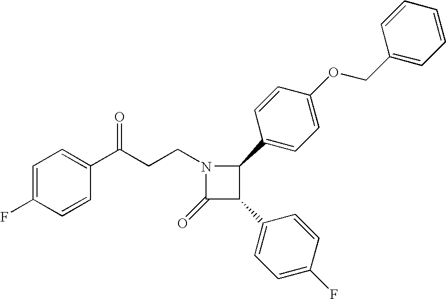

To a solution of 4-fluorophenyl zinc bromide (30.9 mL of 0.5 M solution in THF, 15.4 mmol) at 0° C. was added Pd(PPh₃)₄ (0.85 g, 0.74 mmol) and the solution was stirred for 10 min at 0° C. Subsequently, a solution of 3-[2-(4-benzyloxy-phenyl)-3-(4-fluoro-phenyl)-4-O-azetidin-1-yl]-propionyl chloride (6.44 g, 14.7 mmol) in THF (40 mL) was slowly added at 0° C. The reaction mixture was warmed to 25° C. and stirred at that temperature for an additional 3 hrs before being quenched 1 N HCl. EtOAc was then added and the organic layer was separated, washed with brine, dried (Na₂SO₄) and concentrated. The crude product purified by silica gel chromatography (20→35% EtOAc/hexane) to afford racemic 4-(4-benzyloxy-phenyl)-3-(4-fluoro-phenyl)-1-[3-(4-fluoro-phenyl)-3-O-propyl]-azetidin-2-one (3.14 g, 43%). ¹H-NMR (CDCl₃) δ 7.90-7.86 (m, 2H), 7.41-6.93 (m, 15H), 5.02 (s, 2H), 4.41 (d, 1H), 4.06 (d, 1H), 3.78-3.76 (m, 1H), 3.45-3.32 (m, 2H), 3.18-3.11 (m, 1H); MS (APCI⁺): m/z 498.2 (M+H).

Separation of this racemic product by preparative chiral HPLC provided 4R-(4-benzyloxy-phenyl)-3R-(4-fluoro-phenyl)-1-[3-(4-fluoro-phenyl)-3-O-propyl]-azetidin-2- and 4S-(4-Benzyloxy-phenyl)-3S-(4-fluoro-phenyl)-1-[3-(4-fluoro-phenyl)-3-O-propyl]-azetidin-2-one.

### EXAMPLE 13

### 3-(4-Fluoro-phenyl)-1-[3-(4-fluoro-phenyl)-3-O-propyl]-4-(4-hydroxy-phenyl)-azetidin-2-one

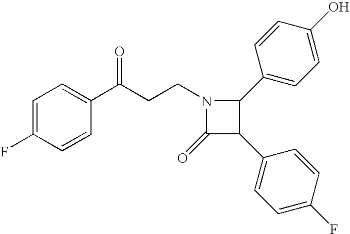

A reaction vessel containing 4-(4-benzyloxy-phenyl)-3-(4-fluoro-phenyl)-1-phenethyl-azetidin-2-one (2.37 g, 4.76 mmol) in EtOH (100 mL) was evacuated and purged with nitrogen. Subsequently, 10% Pd—C (0.2 g) was added and the reaction vessel was charged with hydrogen (50 psi), and the reaction was stirred at 25° C. for 1 hr. Once the reaction was complete as determined by MS, the reaction vessel was purged with nitrogen and the contents were filtered through a celite pad. The filtrate was concentrated and crude product was purified by silica gel chromatography (35→45% EtOAc/hexane) to afford racemic 3-(4-fluoro-phenyl)-1-[3-(4-fluoro-phenyl)-3-O-propyl]-4-(4-hydroxy-phenyl)-azetidin-2-one. ¹H-NMR (CDCl₃) δ 7.90-7.86 (m, 2H), 7.16-6.91 (m, 10H), 6.59 (bs, 1H), 4.41 (d, 1H), 4.06 (d, 1H), 3.82-3.75 (m, 1H), 3.48-3.33 (m, 2H), 3.19-3.10 (m, 1H); MS (APCI⁺): m/z 408.0 (M+H).

### EXAMPLE 14

### 4R-(4-benzyloxy-phenyl)-3R-(4-fluoro-phenyl)-1-[3-(4-fluoro-phenyl)-3S-hydroxy-propyl]-azetidin-2-one

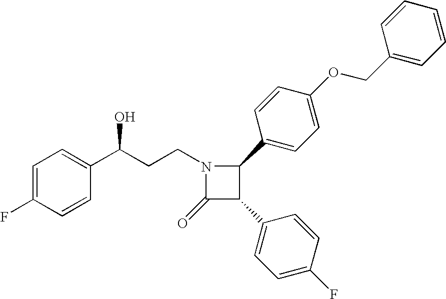

To a solution of 4R-(4-benzyloxy-phenyl)-3R-(4-fluoro-phenyl)-1-[3-(4-fluoro-phenyl)-3-O-propyl]-azetidin-2-one (Example 12, Step E) (0.620 g, 1.25 mmol) in CH₂Cl₂ (50 mL) at −20° C. was added (R)-MeCBS (69 mg, 0.249 mmol). BH₃.SMe₂ (0.14 g, 1.87 mmol) was then added drop-wise at −20° C. The reaction mixture was warmed to 0° C. and stirred for 1 hr. Subsequent TLC analysis indicated that reaction was not complete so it was cooled to −20° C. and a second portion of BH₃.SMe₂ (0.050 g) was added. Reaction was again allowed to warm to 0° C. and stirred for 2 hrs. The reaction was quenched by addition of MeOH. After the solvent was removed under reduced pressure, CH₂Cl₂ was added, and the organic layer was washed with water, dried (Na₂SO₄) and concentrated. Product was purified by silica gel chromatography (35→45% EtOAc/hexane) to provide 4R-(4-benzyloxy-phenyl)-3R-(4-fluoro-phenyl)-1-[3-(4-fluoro-phenyl)-3S-hydroxy-propyl]-azetidin-2-one (0.40 g, 64%). ¹H-NMR (CDCl₃) δ 7.42-7.19 (m, 12H), 7.04-6.94 (m, 5H), 5.05 (s, 2H), 4.69-4.67 (m, 1H), 4.38 (d, 1H), 4.11 (d, 1H), 3.75-3.70 (m, 1H), 3.00-2.90 (m, 2H), 1.88-1.82 (m, 2H); MS (APCl⁻): m/z 534.0 (M−H).

### EXAMPLE 15

### 3R-(4-Fluoro-phenyl)-1-[3-(4-fluoro-phenyl)-3S-hydroxy-propyl]-4R-(4-hydroxy-phenyl)-azetidin-2-one

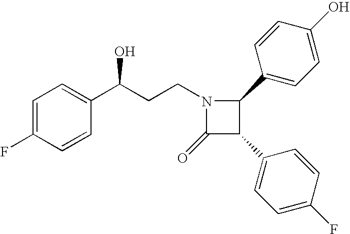

To a solution of 4R-(4-benzyloxy-phenyl)-3R-(4-fluoro-phenyl)-1-[3-(4-fluoro-phenyl)-3S-hydroxy-propyl]-azetidin-2-one (from Example 14) (0.40 g, 0.80 mmol) in MeOH (10 mL) was added Pd—C (20 mg) and ammonium formate (0.25 g, 4.0 mmol). The reaction mixture was acidified to pH 4 via addition of AcOH and was subsequently heated to 50° C. for 3 hrs. A second portion of Pd—C (15 mg) and HCO₂NH₄ (0.20 g, 3.2 mmol) were added after 3 hrs and reaction was then stirred at 50° C. for an additional 3 hrs at which point LC/MS analysis indicated that reaction was complete. Reaction was cooled to 25° C. and filtered through celite. Filtrate was concentrated and EtOAc and water were added. Organic layer was separated, dried (Na₂SO₄), concentrated and purified by silica gel chromatography (55% EtOAc/hexane) to provide 3R-(4-fluoro-phenyl)-1-[3-(4-fluoro-phenyl)-3S-hydroxy-propyl]-4R-(4-hydroxy-phenyl)-azetidin-2-one (0.21 g, 64%). ¹H-NMR (CDCl₃) δ 7.25-7.14 (m, 6H), 7.03-6.94 (m, 4H), 6.86-6.82 (m, 2H), 4.69-4.66 (m, 1H), 4.37 (m, 1H), 4.11 (m, 1H), 3.74-3.71 (m, 1H), 2.97-2.92 (m, 1H), 1.88-1.82 (m, 2H); MS (APCI⁺): m/z 410.0 (M+H).

### Method for Biological Evaluation of Cholesterol Absorption Inhibitors

Male Sprague-Dawley rats (200-400 gm) are maintained in a room with a 12 hour light cycle/12 hour dark cycle for at least one week prior to testing. On the test day the rats are fasted for 8 hours prior to dosing to synchronize initiation of eating once food is presented. Test drug or vehicle is administered by oral gavage approximately 1 hour prior to the start of the dark cycle. One group of animals is dosed with vehicle and given standard chow (chow control), one group is dosed with vehicle and given the same diet supplemented with 5.5% peanut oil, 1.5% cholesterol, and 0.4% cholic acid (PCC diet; PCC control), and the remaining animals are dosed with test agents in vehicle and are given the PCC diet. Animals are given access to their assigned diet ad libitum starting 30 minutes after dosing until study termination 16 hours after drug administration. Animals are euthanized with CO₂, and blood is collected by cardiac puncture for plasma total cholesterol analysis.

Data analysis. Total plasma cholesterol concentrations in chow controls are between about 60 and

about 90 mg/dL and increase to between about 175 and about 240 mg/dL in PCC control animals. The difference in plasma cholesterol between the chow control group and the PCC control group is the elevation caused by the PCC diet. The dose that reduces by 50% the elevation in plasma cholesterol in animals on the PCC diet is the ED₅₀.

Compounds of the invention reduce the elevation in plasma cholesterol by about 50% at doses of between about 30 and about 100 mg/kg in the aforementioned method.

Preferred compounds of the invention reduce the elevation in plasma cholesterol by about 50% at doses less than or equal to 30 mg/kg in the aforementioned method.

### EXAMPLE 16

### 4S-(4-Benzyloxy-phenyl)-3S-(4-fluoro-phenyl)-1-[3-(4-fluoro-phenyl)-3S-hydroxy-propyl]-azetidin-2-one

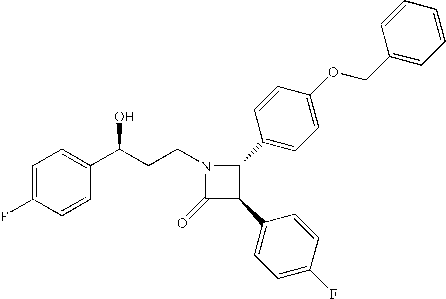

Prepared from 4S-(4-benzyloxy-phenyl)-3S-(4-fluoro-phenyl)-1-[3-(4-fluoro-phenyl)-3-O-propyl]-azetidin-2-one (from Example 12) using the method of Example 14. MS (APCI⁺): m/z 534.1 (M+H).

### EXAMPLE 17

### 3S-(4-Fluoro-phenyl)-1-[3-(4-fluoro-phenyl)-3S-hydroxy-propyl]-4S-(4-hydroxy-phenyl)-azetidin-2-one

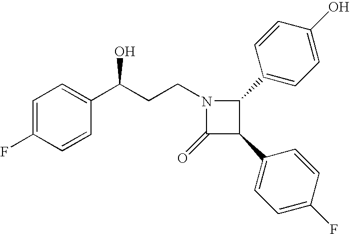

Prepared from 4S-(4-benzyloxy-phenyl)-3S-(4-fluoro-phenyl)-1-[3-(4-fluoro-phenyl)-3S-hydroxy-propyl]-azetidin-2-one (from Example 16) in a manner analogous to that of Example 15. MS(APCI⁺): m/z 410.2 (M+H).

What is claimed is:

1. A compound of the formula (1) 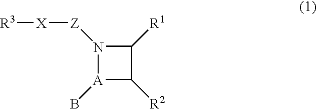
or a pharmaceutically acceptable salt, ester, hydrate, amide, or stereoisomer thereof, wherein
A-B is C═O, C═S, SO, or SO₂;
X is a C₁-C₃ alkylene optionally containing a double or triple bond, or a C₁-C₃ heteroalkylene, wherein the C₁-C₃ alkylene or C₁-C₃ heteroalkylene is unsubstituted or substituted on carbon atoms with 0, 1, or 2 substituents selected from the group consisting of C₁-C₆ alkyl, ═O, —C(O)Rₐ, —OR_{b}, R_{c}, —OC(O)R_{d}, —NR′R″, halo, C₃-C₆ cycloalkyl, C₃-C₆ heterocycloalkyl, aryl, heteroaryl, and cyano; wherein
Rₐ is hydroxy, —OC₁-C₆ alkyl or C₁-C₆ alkyl;
R_{b} is hydrogen, SO₃H, PO₃H, C₁-C₆ alkyl, or C₁-C₆ aralkyl;
R_{c} is YG; wherein Y is NR′, S, or O;
R_{d} is NR′R″, C₁-C₆ alkyl, C₁-C₆ aralkyl, C₃-C₆ cycloalkyl, C₃-C₆ heterocycloalkyl, aryl, or heteroaryl;
R′ and R″ are each independently selected from the group consisting of hydrogen and C₁-C₆ alkyl;
Z is a C₁-C₂ alkylene optionally substituted with 0, 1, or 2 substituents selected from the group consisting of C₁-C₆ alkyl, ═O, —C(O)Rₐ, —OR_{b}, —OC(O)R_{d}, —NR′R″, halo, C₃-C₆ cycloalkyl,
C₃-C₆ heterocycloalkyl, aryl, heteroaryl, and cyano;
R¹ is aryl or heteroaryl optionally substituted with one to three substituents independently selected from the group consisting of halo, —C(O)Rₐ, —OR_{b}, R_{c}, C₁-C₂₀ alkyl, C₁-C₆ aralkyl, and cyano;
R² is C₁-C₆ alkyl, C₃-C₆ cycloalkyl, C₃-C₆ heterocycloalkyl, aryl, heteroaryl, or C₁-C₆ aralkyl, wherein said
C₁-C₆ alkyl, C₃-C₆ cycloalkyl, C₃-C₆ heterocycloalkyl, aryl, heteroaryl, or C₁-C₆ aralkyl groups are optionally substituted with one to three substituents independently selected from the group consisting of halo,
—C(O)Rₐ, —OR_{b}, C₁-C₂₀ alkyl, and cyano; and
R³ is C₃-C₆ cycloalkyl, C₃-C₆ heterocycloalkyl, aryl or heteroaryl, wherein the C₃-C₆ cycloalkyl,
C₃-C₆ heterocycloalkyl, aryl or heteroaryl groups are optionally substituted with one to three substituents independently selected from the group consisting of halo, —C(O)R^{a}, —OR^{b}, C₁-C₂₀ alkyl, C₁-C₆ alkyl-NR′R″, and cyano; and
G is selected from the group consisting of hydrogen, 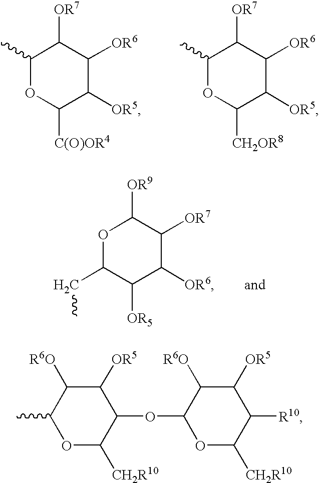
wherein 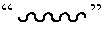
indicates the point of attachment and wherein R⁴, R⁵, R⁶, R⁷, R⁸, and R⁹ are each independently selected from the group consisting of hydrogen, C₁-C₆ alkyl, C₁-C₆ aralkyl, —C(O)C₁-C₆ alkyl,
—C(O)aryl, and aryl; and R¹⁰ is selected from the group consisting of hydrogen, hydroxy, C₁-C₆ alkyl,
—OC₁-C₆ alkyl, and NR′R″.

2. The compound of claim 1 wherein R¹ is aryl optionally substituted with one to three substituents independently selected from the group consisting of halo, —C(O)Rₐ, —OR_{b}, R_{c}, C₁-C₆ alkyl, and C₁-C₆ aralkyl.

3. The compound of claim 2 wherein R² is aryl optionally substituted with one to three substituents independently selected from the group consisting of halo, —C(O)Rₐ, —OR_{b}, and C₁-C₆ alkyl.

4. The compound of claim 3 wherein R³ is aryl optionally substituted with one to three substituents independently selected from the group consisting of halo, —C(O)Rₐ, —OR_{b}, C₁-C₆ alkyl, and
C₁-C₆ alkyl-NR′R″.

5. The compound of claim 1 wherein
A-B is C═O;
X is a C₁-C₃ alkylene or C₁-C₃ heteroalkylene, wherein the C₁-C₃ alkylene or C₁-C₃ heteroalkylene is optionally substituted on carbon atoms with 0, 1, or 2 substituents selected from the group consisting of C₁-C₆ alkyl, ═O, —C(O)Rₐ, —OR_{b}, —OC(O)R_{d}, and halo;
Z is a C₁-C₂ alkylene optionally substituted with 0, 1, or 2 substituents selected from the group consisting of C₁-C₆ alkyl, ═O, C(O)Rₐ, OR_{b}, OC(O)R_{d}, halo, aryl, heteroaryl, and NR′R″;
R¹ is aryl optionally substituted with one to three substituents independently selected from the group consisting of halo, —C(O)Rₐ, —OR_{b}, R_{c}, C₁-C₆ alkyl, and C₁-C₆ aralkyl;
R² is aryl optionally substituted with one to three substituents independently selected from the group consisting of halo, —C(O)Rₐ, —OR_{b}, and C₁-C₆ alkyl; and
R³ is aryl optionally substituted with one to three substituents independently selected from the group consisting of halo, —C(O)Rₐ, —OR_{b}, and C₁-C₆ alkyl.

6. The compound of claim 5 wherein
Z is a C₁-C₂ alkylene optionally substituted with 0, 1, or 2 substituents selected from the group consisting of C₁-C₆ alkyl, ═O, halo, —C(O)Rₐ, —OR_{b}, —OC(O)R_{d}, and aryl.

7. The compound of claim 5 wherein
X is a C₁-C₃ alkylene optionally substituted with 0, 1, or 2 substituents selected from the group consisting of C₁-C₆ alkyl, ═O, —C(O)Rₐ, —OR_{b}, —OC(O)R_{d}, and halo; and
Z is a C₁-C₂ alkylene optionally substituted with 0, 1, or 2 substituents selected from the group consisting of C₁-C₆ alkyl, ═O, and —OR_{b}.

8. The compound of claim 1 wherein
A-B is C═O;
X is a C₁-C₃ alkylene that is unsubstituted or substituted with 0, 1, or 2 substituents selected from the group consisting of C₁-C₆ alkyl, ═O, and —OR_{b};
Z is a C₁-C₂ alkylene optionally substituted with 0, 1, or 2 substituents selected from the group consisting of ═O and —OR_{b};
R¹ is phenyl optionally substituted with —OR_{b};
R² is phenyl optionally substituted with a substituent independently selected from the group consisting of halo and —OR_{b}; and
R³ is phenyl optionally substituted with a substituent independently selected from the group consisting of halo and —OR_{b}.

9. A compound selected from the group consisting of:
(3R,4R)-bis-(4-methoxy-phenyl)-1-(3-phenyl-propyl)-azetidin-2-one;
(3S,4S)-bis-(4-methoxy-phenyl)-1-(3-phenyl-propyl)-azetidin-2-one;
3R-(4-Fluoro-phenyl)-4R-(4-methoxy-phenyl)-1-(3-phenyl-propyl)-azetidin-2-one;
4-(4-Benzyloxy-phenyl)-3-(4-fluoro-phenyl)-1-phenethyl-azetidin-2-one;
3R-(4-Fluoro-phenyl)-4R-(4-hydroxy-phenyl)-1-phenethyl-azetidin-2-one;
3S-(4-Fluoro-phenyl)-4S-(4-hydroxy-phenyl)-1-phenethyl-azetidin-2-one;
3R-(4-Fluoro-phenyl)-4R-(4-hydroxy-phenyl)-1-(3-phenyl-propyl)-azetidin-2-one;
3S-(4-Fluoro-phenyl)-4S-(4-hydroxy-phenyl)-1-(3-phenyl-propyl)-azetidin-2-one;
3R-(4-Fluoro-phenyl)-4R-(4-hydroxy-phenyl)-1-(4-phenyl-butyl)-azetidin-2-one;
3S-(4-Fluoro-phenyl)-4S-(4-hydroxy-phenyl)-1-(4-phenyl-butyl)-azetidin-2-one;
4-(4-Benzyloxy-phenyl)-3-(4-fluoro-phenyl)-1-(4-phenyl-butyl)-azetidin-2-one;
4R-(4-Benzyloxy-phenyl)-3R-(4-fluoro-phenyl)-1-[3-(4-fluoro-phenyl)-3-O-propyl]-azetidin 2-one;
3-(4-Fluoro-phenyl)-1-[3-(4-fluoro-phenyl)-3-O-propyl]-4-(4-hydroxy-phenyl)-azetidin-2-one;
4R-(4-Benzyloxy-phenyl)-3R-(4-fluoro-phenyl)-1-[3-(4-fluoro-phenyl)-3S-hydroxy-propyl]-azetidin-2-one;
3R-(4-Fluoro-phenyl)-1-[3-(4-fluoro-phenyl)-3S-hydroxy-propyl]-4R-(4-hydroxy-phenyl)-azetidin-2-one;
4S-(4-Benzyloxy-phenyl)-3S-(4-fluoro-phenyl)-1-[3-(4-fluoro-phenyl)-3S-hydroxy-propyl]-azetidin-2-one;
3S-(4-Fluoro-phenyl)-1-[3-(4-fluoro-phenyl)-3S-hydroxy-propyl]-4S-(4-hydroxy-phenyl)-azetidin-2-one; and
and pharmaceutically acceptable salts, esters, amides, hydrates, and stereoisomers thereof.

10. A compound of formula (2) or (3) 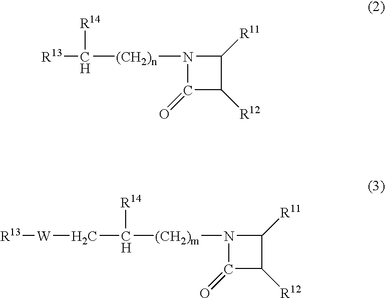
or a pharmaceutically acceptable salt, ester, hydrate, amide, or stereoisomer thereof, wherein
n is 1, 2, 3, or 4;
m is 1 or 2;
W is O, NR′R″ or S;
R¹¹ is phenyl optionally substituted with one to three substituents independently selected from the group consisting of halo, —C(O)Rₐ, —OR_{b}, R_{c}, C₁-C₂₀ alkyl, C₁-C₆ aralkyl, and cyano;
R¹² is C₁-C₆ alkyl, C₃-C₆ cycloalkyl, C₃-C₆ heterocycloalkyl, aryl, heteroaryl, or C₁-C₆ aralkyl, wherein said C₁-C₆ alkyl, C₃-C₆ cycloalkyl, C₃-C₆ heterocycloalkyl, aryl, heteroaryl, or C₁-C₆ aralkyl groups are optionally substituted with one to three substituents independently selected from the group consisting of halo, —C(O)Rₐ, —OR_{b}, C₁-C₂₀ alkyl, and cyano;
R¹³ is aryl or heteroaryl, wherein the aryl or heteroaryl groups are optionally substituted with one to three substituents independently selected from the group consisting of halo, —C(O)Rₐ, —OR_{b}, C₁-C₂₀ alkyl, and C₁-C₆ alkyl-NR′R″; and
R¹⁴ is selected from the group consisting of C₁-C₆ alkyl, ═O, C(O)Rₐ, OR_{b}, R_{c}, OC(O)R_{d}, —NR′R″, halo,
C₃-C₆ cycloalkyl, C₃-C₆ heterocycloalkyl, aryl, heteroaryl, and cyano; wherein
Rₐ is hydroxy, —OC₁-C₆ alkyl or C₁-C₆ alkyl;
R_{b} is hydrogen, SO₃H, PO₃H, C₁-C₆ alkyl, or C₁-C₆ aralkyl;
R^{c} is YG; wherein Y is NR′, S, or O;
R_{d} is NR′R″, C₁-C₆ alkyl, C₁-C₆ aralkyl, C₃-C₆ cycloalkyl, C₃-C₆ heterocycloalkyl, aryl, or heteroaryl;
R′ and R″ are each independently selected from the group consisting of hydrogen and C₁-C₆ alkyl;
G is selected from the group consisting of hydrogen, 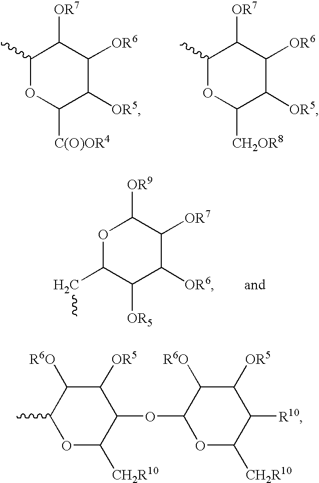 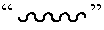
indicates the point of attachment, R⁴, R⁵, R⁶, R⁷, R⁸, and R⁹ are each independently selected from the group consisting of hydrogen, C₁-C₆ alkyl, C₁-C₆ aralkyl, —C(O)C₁-C₆ alkyl, —C(O)aryl, and aryl; and R¹⁰ is selected from the group consisting of hydrogen, hydroxy, C₁-C₆ alkyl, —OC₁-C₆ alkyl, and NR′R″.
